(19) United States
(12) Patent Application Publication
(10) Pub. No.: US 2021/0068742 A1
(43) Pub. Date: Mar. 11, 2021

(54) IMAGE PROCESSING SYSTEM, IMAGING APPARATUS, ELECTRONIC DEVICE, METHODS OF CONTROLLING THE SYSTEM, AND THE APPARATUSES, AND STORAGE MEDIUM

(71) Applicant: CANON KABUSHIKI KAISHA, Tokyo (JP)

(72) Inventors: Atsushi Goto, Kanagawa (JP); Takashi Sugimoto, Kanagawa (JP); Yoshikazu Kawai, Tokyo (JP); Yosato Hitaka, Tokyo (JP); Tomoki Kuroda, Tokyo (JP)

(21) Appl. No.: 17/103,575

(22) Filed: Nov. 24, 2020

Related U.S. Application Data
(63) Continuation of application No. PCT/JP2019/021094, filed on May 28, 2019.

(30) Foreign Application Priority Data
May 31, 2018 (JP) 2018-104922
Feb. 5, 2019 (JP) 2019-018653
May 22, 2019 (JP) 2019-095938

(51) Int. Cl. A61B 5/00; G16H 50/20; G16H 10/60; G16H 40/63; G06T 7/00

(52) U.S. Cl. CPC A61B 5/445; G16H 50/20; A61B 5/0077; G06T 2207/30088; G16H 40/63; G06T 7/0012; G16H 10/60

(57) ABSTRACT

An image processing system comprises an imaging apparatus and an image processing apparatus. The imaging apparatus receives light from a subject to generate image data, outputs the image data to a communication network, and displays an image based on the image data. The image processing apparatus acquires the image data over the communication network, and extracts an affected area of the subject from the image data. The image processing apparatus outputs information indicating a result of extraction of the affected area to the communication network, and the imaging apparatus acquires the information indicating the result of extraction of the affected area. The imaging apparatus performs display based on the information and causes a user to input evaluation values of a plurality of predetermined evaluation items in the affected area.

### CROSS-REFERENCE TO RELATED APPLICATIONS

This application is a Continuation of International Patent Application No. PCT/JP2019/021094, filed May 28, 2019, which claims the benefit of Japanese Patent Application No. 2018-104922, filed May 31, 2018, Japanese Patent Application No. 2019-018653, filed Feb. 5, 2019, and Japanese Patent Application No. 2019-095938, filed May 22, 2019, those of which are hereby incorporated by reference herein in their entirety.

### BACKGROUND OF THE INVENTION

### Field of the Invention

The present invention relates to a technology to evaluate a certain area of a subject from an image.

### Background Art

In a state in which a person or an animal lies down, a contact region between the body and a floor, a mat, or a mattress below the body is compressed by the body weight.

If the same posture is continued, vascular insufficiency occurs in the contact region between the floor and the body to cause necrosis of the surrounding tissue. The state in which the tissue necrosis occurs is called pressure ulcer or bedsore. It is necessary to give pressure ulcer care, such as body pressure dispersion and skin care, to the patient developing the pressure ulcer to periodically evaluate and manage the pressure ulcer.

Measurement of the size of the pressure ulcer is known as one method of evaluating the pressure ulcer.

For example, DESIGN-R (registered trademark), which is an evaluation index of the pressure ulcer developed by Academic Education Committee of the Japanese Society of Pressure Ulcers, is known as an example in which the size of the pressure ulcer is used in the evaluation, as described in NPL1.

The DESIGN-R (registered trademark) is a tool to evaluate the healing process of a wound, such as the pressure ulcer. This tool is named from the initial letters of evaluation items: Depth, Exudate, Size, Inflammation/Infection, Granulation, and Necrotic tissue. Pocket is also included in the evaluation items, in addition to the above evaluation items, although the initial letter of Pocket is not used in the name.

The DESIGN-R (registered trademark) is classified into two groups for classification of the severity level, which is used for common and simple evaluation, and for process evaluation in which the flow of the healing process is indicated in detail. In the DESIGN-R (registered trademark) for classification of the severity level, the six evaluation items are classified into two: mild and severe. The mild evaluation items are represented using the lowercase letters alphabet and the severe evaluation items are represented using the capital letters alphabet.

The evaluation using the DESIGN-R (registered trademark) for classification of the severity level in first treatment enables the rough state of the pressure ulcer to be figured out. Since the item having a problem is revealed, it is possible to easily determine the treatment policy.

The DESIGN-R (registered trademark) capable of comparison of the severity level between patients, in addition to the process evaluation, is also defined as the DESIGN-R (registered trademark) for process evaluation. Here, R represents Rating (evaluation and rating). Different weights are added to the respective items and the sum (0 points to 66 points) of the weights of the six items excluding the depth represents the severity level of the pressure ulcer. With the DESIGN-R (registered trademark), it is possible to objectively evaluate the course of treatment in detail after the treatment is started to enable the comparison of the severity level between the patients, in addition to the evaluation of the course of an individual.

In the evaluation of the size in the DESIGN-R (registered trademark), the major axis length (cm) and the minor axis length (the maximum diameter orthogonal to the major axis length) (cm) of a skin injury range are measured and the size, which is the numerical value given by multiplying the major axis length by the minor axis length, is classified into seven stages. The seven stages include s0: no skin injury, s3: lower than four, s6: not lower than four and lower than 16, s8: not lower than 16 and lower than 36, s9: not lower than 36 and lower than 64, s12: not lower than 64 and lower than 100, and s15: not lower than 100.

Currently, the evaluation of the size of the pressure ulcer is often based on the value resulting from manual measurement of an affected area using a measure. Specifically, the maximum straight-line distance between two points in the skin injury range is measured and the measured distance is used as the major axis length. The length orthogonal to the major axis length is used as the minor axis length, and the value given by multiplying the major axis length by the minor axis length is set as the size of the pressure ulcer.

### CITATION LIST

### Non Patent Literature

NPL1: Pressure ulcer Guidebook Edition 2, Pressure ulcer prevention & management Guideline (Edition 4) Compliance, Edited by Japanese Society of Pressure Ulcers, ISBN13 978-4796523608, Shourin-sha, pp. 23.

However, the pressure ulcer often has a complicated shape and it is necessary to adjust the usage of the measure in the manual evaluation of the size of the pressure ulcer. Since it is necessary to perform the above work at least two times to measure the major axis length and the minor axis length, it takes a time and a heavy workload is required. In addition, since the patient the pressure ulcer of whom is to be evaluated is required to keep the same posture during the work, the manual evaluation of the size of the pressure ulcer is considered to impose a heavy burden on the patient.

It is recommended to perform the rating once per week or two weeks in the DESIGN-R (registered trademark) and it is necessary to perform the measurement repeatedly. In addition, the position to be determined to be the major axis length of the pressure ulcer may be varied depending on the individuals in the manual measurement and it is difficult to ensure the accuracy of the measurement.

Although the example is described above in which the evaluation of the pressure ulcer is performed based on the DESIGN-R (registered trademark), the above description is not limited to the case of the DESIGN-R (registered trademark) and similar problems occur regardless of the method of measuring the size of the pressure ulcer. It is necessary to perform the manual measurement for multiple places to calculate the area of the pressure ulcer and, thus, the workload is caused.

As another problem, the evaluation items of the pressure ulcer include the evaluation items that are desirably visually determined, in addition to the evaluation items including the size which are measured. The evaluation items that should be visually determined are subsequently input by an evaluator onto an electronic health record or a paper medium while watching image data that is captured. In this case, since the input device used for information indicating the size is different from the input device used for other information, the input operation is made complicated and omission is likely to occur.

These problems are not limited to the pressure ulcer and similar problems occur for an affected area, such as a burn injury or a laceration, on the body surface.

### SUMMARY OF THE INVENTION

An image processing system of one aspect of the present invention includes an image processing system comprising an imaging apparatus and an image processing apparatus, wherein: the imaging apparatus includes an imaging device configured to receive light from a subject to generate image data, a first communication circuit configured to output the image data to a communication network, and a display configured to display an image based on the image data generated by the imaging device, the image processing apparatus includes a second communication circuit configured to acquire the image data over the communication network, and an arithmetic circuit configured to extract an affected area of the subject from the image data, the second communication circuit outputs information indicating a result of extraction of the affected area extracted by the arithmetic circuit to the communication network, the first communication circuit acquires the information indicating the result of extraction of the affected area over the communication network, and the display performs display based on the information indicating the result of extraction of the affected area and display causing a user to input evaluation values of a plurality of predetermined evaluation items in the affected area.

An imaging apparatus of another aspect of the present invention includes an imaging apparatus comprising: an imaging device configured to receive light from a subject to generate image data; a communication circuit configured to output the image data to an external apparatus over a communication network; and a display configured to display an image based on the image data generated by the imaging device, wherein the communication circuit acquires information indicating a result of extraction of an affected area of the subject in the image data from the external apparatus over the communication network, and the display performs display based on the information indicating the result of extraction of the affected area and display causing a user to input evaluation values of a plurality of predetermined evaluation items in the affected area.

A method of controlling an image processing system of another aspect of the present invention includes a method of controlling an image processing system including an imaging apparatus that includes an imaging device, a display, and first communication device and an image processing apparatus that includes an arithmetic device and a second communication device, the method comprising: receiving light from a subject to generate image data by the imaging device; outputting the image data to a communication network by the first communication device; acquiring the image data over the communication network by the second communication device; extracting an affected area of the subject from the image data by the arithmetic device; outputting information indicating a result of extraction of the affected area to the communication network by the second communication device; acquiring the information indicating the result of extraction of the affected area over the communication network by the first communication device; and performing display based on the information indicating the result of extraction of the affected area and display causing a user to input evaluation values of a plurality of predetermined evaluation items in the affected area by the display.

A method of controlling an imaging apparatus of another aspect of the present invention includes the method comprising: receiving light from a subject to generate image data; outputting the image data to an external apparatus over a communication network; acquiring information indicating a result of extraction of an affected area of the subject in the image data from the external apparatus over the communication network; and causing a display to perform display based on the information indicating the result of extraction of the affected area and display causing a user to input evaluation values of a plurality of predetermined evaluation items in the affected area.

A computer-readable non-volatile storage medium of another aspect of the present invention includes a computer-readable non-volatile storage medium storing an instruction causing a computer to perform steps of a method of controlling an imaging apparatus, the method of controlling the imaging apparatus comprising: receiving light from a subject to generate image data; outputting the image data to an external apparatus over a communication network; acquiring information indicating a result of extraction of an affected area of the subject in the image data from the external apparatus over the communication network; and causing a display to perform display based on the information indicating the result of extraction of the affected area and display causing a user to input evaluation values of a plurality of predetermined evaluation items in the affected area.

An image apparatus of another aspect of the present invention includes an imaging apparatus comprising: an imaging device configured to receive light from a subject to generate image data; a control circuit configured to acquire a result of extraction of a certain area of the subject in the image data; and an interface circuit configured to cause a user to input evaluation values of a plurality of predetermined evaluation items in the certain area of the subject, wherein the control circuit associates the evaluation values of the input plurality of evaluation items with the image data.

A method of controlling an imaging apparatus of another aspect of the present invention includes a method of controlling an imaging apparatus of another aspect of the present invention includes a method of controlling an imaging apparatus, the method comprising: receiving light from a subject to generate image data; acquiring a result of extraction of a certain area of the subject in the image data; causing a user to input evaluation values of a plurality of predetermined evaluation items in the certain area of the subject; and associating the evaluation values of the input plurality of evaluation items with the image data.

A computer-readable non-volatile storage medium of another aspect of the present invention includes a computer-readable non-volatile storage medium storing an instruction causing a computer to perform steps of a method of controlling an imaging apparatus, the method of controlling the imaging apparatus comprising: receiving light from a subject to generate image data; acquiring a result of extraction of a certain area of the subject in the image data; causing a user to input evaluation values of a plurality of predetermined evaluation items in the certain area of the subject; and associating the evaluation values of the input plurality of evaluation items with the image data.

An electronic device of another aspect of the present invention includes an electronic device comprising: a communication circuit configured to acquire image data generated by an imaging apparatus and information indicating evaluation values of a plurality of evaluation items for an affected area of a subject in the image data, which is input by a user with the imaging apparatus, over a communication network; and a control circuit configured to cause a display to display an image based on the image data and the evaluation values of the plurality of evaluation items.

A method of controlling an electronic device of another aspect of the present invention includes a method of controlling an electronic device, the method comprising: acquiring image data generated by an imaging apparatus and information indicating evaluation values of a plurality of evaluation items for an affected area of a subject in the image data, which is input by a user with the imaging apparatus, over a communication network; and causing a display to display an image based on the image data and the evaluation values of the plurality of evaluation items.

A computer-readable non-volatile storage medium of another aspect of the present invention includes a computer-readable non-volatile storage medium storing an instruction causing a computer to perform steps of a method of controlling an electronic device, the method of controlling the electronic device is characterized by comprising: acquiring image data generated by an imaging apparatus and information indicating evaluation values of a plurality of evaluation items for an affected area of a subject in the image data, which is input by a user with the imaging apparatus, over a communication network; and causing a display to display an image based on the image data and the evaluation values of the plurality of evaluation items.

Further features of the present invention will become apparent from the following description of exemplary embodiments with reference to the attached drawings.

### BRIEF DESCRIPTION OF THE DRAWINGS

FIG. 1 is a diagram schematically illustrating an image processing system according to a first embodiment.

FIG. 2 is a diagram illustrating an example of the hardware configuration of an imaging apparatus included in the image processing system.

FIG. 3 is a diagram illustrating an example of the hardware configuration of an image processing apparatus included in the image processing system.

FIG. 4 is a work flow chart illustrating the operation of the image processing system according to the first embodiment.

FIG. 5 is a diagram for describing how the area of an area is calculated.

FIG. 6A is a diagram for describing image data including an affected area.

FIG. 6B is a diagram for describing how information indicating the result of extraction of the affected area and information indicating the size of the affected area are superimposed on the image data.

FIG. 7A is a diagram for describing a method of superimposing the information indicating the result of extraction of the affected area and information that includes a major axis length and a minor axis length of the affected area and that indicates the size of the affected area on the image data.

FIG. 7B is a diagram for describing another method of superimposing the information indicating the result of extraction of the affected area and the information that includes the major axis length and the minor axis length of the affected area and that indicates the size of the affected area on the image data.

FIG. 7C is a diagram for describing another method of superimposing the information indicating the result of extraction of the affected area and the information that includes the major axis length and the minor axis length of the affected area and that indicates the size of the affected area on the image data.

FIG. 8A is a diagram for describing a method of causing a user to input information about a region of the affected area.

FIG. 8B is a diagram for describing the method of causing the user to input the information about the region of the affected area.

FIG. 8C is a diagram for describing a method of causing the user to input information about an evaluation value of the affected area.

FIG. 8D is a diagram for describing the method of causing the user to input the information about the evaluation value of the affected area.

FIG. 8E is a diagram for describing the method of causing the user to input the information about the evaluation value of the affected area.

FIG. 8F is a diagram for describing another method of causing the user to input the information about the region of the affected area.

FIG. 8G is a diagram for describing another method of causing the user to input the information about the region of the affected area.

FIG. 9 is a work flow chart illustrating the operation of an image processing system according to a second embodiment.

FIG. 10 is a diagram schematically illustrating an image processing system according to a third embodiment.

FIG. 11 is a work flow chart illustrating the operation of the image processing system according to the third embodiment.

FIG. 12A is a diagram for describing a method of displaying the information about the region of the affected area for which the evaluation value has been acquired.

FIG. 12B is a diagram for describing a method of displaying the information about the evaluation value of the affected area, which has been acquired.

FIG. 13 is a diagram for describing an example of a data selection window displayed in a browser of a terminal apparatus.

FIG. 14 is a diagram for describing an example of a data browsing window displayed in the browser of the terminal apparatus.

FIG. 15 is a work flow chart illustrating a modification of the operation of the image processing system according to the third embodiment.

### DESCRIPTION OF THE EMBODIMENTS

An object of the embodiments is to improve the user-friendliness in evaluation of a certain area of a subject.

Exemplary embodiments of the present invention will herein be described in detail with reference to the drawings.

### First Embodiment

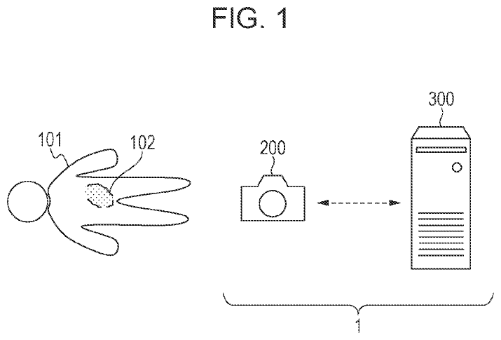

An image processing system according to an embodiment of the present invention will now be described with reference to FIG. 1 to FIG. 3. FIG. 1 is a diagram schematically illustrating an image processing system 1 according to a first embodiment. The image processing system 1 is composed of an imaging apparatus 200, which is a portable handheld device, and an image processing apparatus 300. In the present embodiment, an example of a clinical condition of an affected area 102 of a subject 101 is described as the pressure ulcer over the hip.

In the image processing system 1 according to the embodiment of the present invention, the imaging apparatus 200 shoots the affected area 102 of the subject 101, acquires a subject distance, and transmits the data to the image processing apparatus 300. The image processing apparatus 300 extracts the affected area from the received image data, measures the area per one pixel of the image data based on the information including the subject distance, and measures the area of the affected area 102 from the result of extraction of the affected area 102 and the area per one pixel. Although the example is described in the present embodiment in which the affected area 102 is the pressure ulcer, the affected area 102 is not limited to this and may be a burn injury or a laceration.

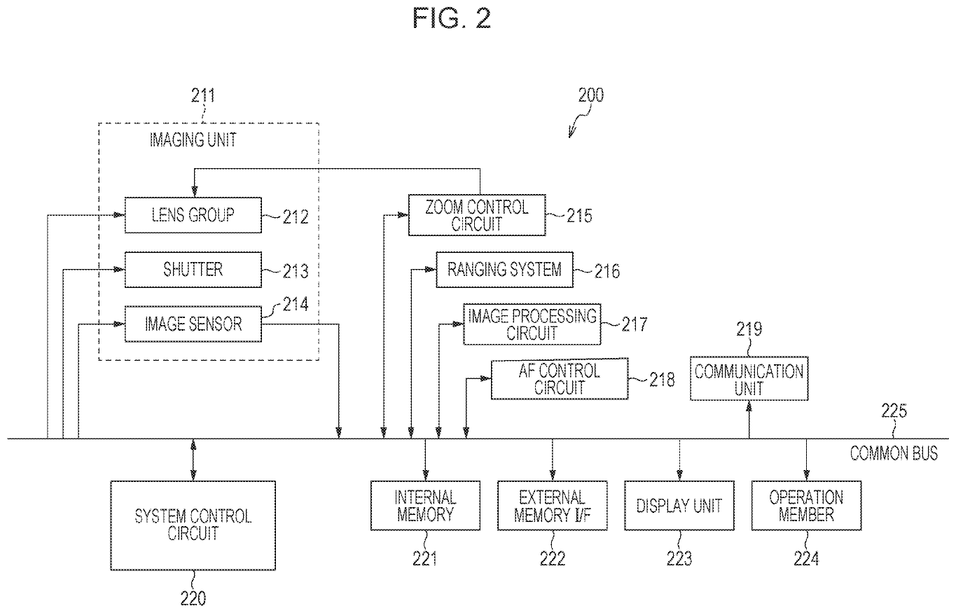

FIG. 2 is a diagram illustrating an example of the hardware configuration of the imaging apparatus 200 included in the image processing system 1. For example, a common single-lens camera, a compact digital camera, or a smartphone or a tablet provided with a camera having an automatic focus function may be used as the imaging apparatus 200.

An imaging unit 211 includes a lens group 212, a shutter 213, and an image sensor 214. Changing the positions of multiple lenses included in the lens group 212 enables the focus position and the zoom magnification to be varied. The lens group 212 also includes a diaphragm for adjusting the amount of exposure.

The image sensor 214 is composed of a charge-storage-type solid-state image sensor, such as a charge coupled device (CCD) or a complementary metal oxide semiconductor (CMOS) sensor, which converts an optical image into image data. An image is formed on the image sensor 214 from reflected light from the subject through the lens group 212 and the shutter 213. The image sensor 214 generates an electrical signal corresponding to the subject image and outputs the image data based on the electrical signal.

The shutter 213 performs exposure and light shielding to the image sensor 214 by opening and closing a shutter blade member to control the exposure time of the image sensor 214. An electronic shutter that controls the exposure time in response to driving of the image sensor 214 may be used, instead of the shutter 213. When the electronic shutter is operated using the CMOS sensor, a reset process is performed to set the accumulation of charge of the pixel to zero for each pixel or for each area (for example, for each line) composed of multiple pixels. Then, a scanning process is performed to read out a signal corresponding to the accumulation of charge after a predetermined time for each pixel or area for which the reset process is performed.

A zoom control circuit 215 controls a motor (not illustrated) for driving a zoom lens included in the lens group 212 to control the optical magnification of the lens group 212. The lens group 212 may be a single focus lens group without a zoom function. In this case, it is not necessary to provide the zoom control circuit 215.

A ranging system 216 calculates distance information to the subject. A common phase-difference-type ranging sensor installed in a single-lens reflex camera may be used as the ranging system 216 or a system using a time of flight (TOF) sensor may be used as the ranging system 216. The TOF sensor is a sensor that measures the distance to an object based on the time difference (or the phase difference) between the timing when irradiation waves are transmitted and the timing when reflected waves resulting from reflection of the irradiation waves from the object are received. In addition, for example, a position sensitive device (PSD) method using the PSD as a photo detector may be used for the ranging system.

Alternatively, the image sensor 214 may have a configuration which includes multiple photoelectric conversion areas for each pixel and in which the pupil positions corresponding to the multiple photoelectric conversion areas included in a common pixel are varied. With this configuration, the ranging system 216 is capable of calculating the distance information for each pixel or for each area position from the phase difference between the images which are output from the image sensor 214 and which are acquired from the photoelectric conversion areas corresponding to the respective pupil areas.

The ranging system 216 may have a configuration in which the distance information in a predetermined one or multiple raging areas in the image is calculated or may have a configuration in which a distance map indicating the distribution of the pieces of distance information in multiple pixels or areas in the image is acquired.

Alternatively, the ranging system 216 may perform TV-auto focus (AF) or contrast AF, in which the radio-frequency components of the image data are extracted for integration and the position of a focus lens having the maximum integration value is determined, to calculate the distance information from the position of the focus lens.

An image processing circuit 217 performs predetermined image processing to the image data output from the image sensor 214. The image processing circuit 217 performs a variety of image processing, such as white balance adjustment, gamma correction, color interpolation, demosaicing, and filtering, to image data output from the imaging unit 211 or image data recorded in an internal memory 221. In addition, the image processing circuit 217 performs a compression process to the image data subjected to the image processing according to, for example, Joint Photographic Experts Group (JPEG) standard.

An AF control circuit 218 determines the position of the focus lens included in the lens group 212 based on the distance information calculated in the ranging system 216 to control a motor that drives the focus lens.

A communication unit 219 is a wireless communication module used by the imaging apparatus 200 to communicate with an external device, such as the image processing apparatus 300, over a wireless communication network (not illustrated). A specific example of the network is a network based on Wi-Fi standard. The communication using the Wi-Fi may be realized using a router. The communication unit 219 may be realized by a wired communication interface, such as universal serial bus (USB) or local area network (LAN).

A system control circuit 220 includes a central processing unit (CPU) and controls the respective blocks in the imaging apparatus 200 in accordance with programs stored in the internal memory 221 to control the entire imaging apparatus 200. In addition, the system control circuit 220 controls the imaging unit 211, the zoom control circuit 215, the ranging system 216, the image processing circuit 217, the AF control circuit 218, and so on. The system control circuit 220 may use a field programmable gate array (FPGA), an application specific integrated circuit (ASIC), or the like, instead of the CPU.

The internal memory 221 is composed of a rewritable memory, such as a flash memory or a synchronous dynamic random access memory (SDRAM). The internal memory 221 temporarily stores a variety of setup information including information about the point of focus and the zoom magnification in image capturing, which is necessary for the operation of the imaging apparatus 200, the image data captured by the imaging unit 211, and the image data subjected to the image processing in the image processing circuit 217. The internal memory 221 may temporarily record, for example, the image data and analysis data including information indicating the size of the subject, which are received through the communication with the image processing apparatus 300 by the communication unit 219.

An external memory interface (I/F) 222 is an interface with a non-volatile storage medium, such as a secure digital (SD) card or a compact flash (CF) card, which is capable of being loaded in the imaging apparatus 200. The external memory I/F 222 records the image data processed in the image processing circuit 217 and the image data, the analysis data, and so on received through the communication with the image processing apparatus 300 by the communication unit 219 on the storage medium, which is capable of being loaded in the imaging apparatus 200. The external memory I/F 222 may read out the image data recorded on the storage medium, which is capable of being loaded in the imaging apparatus 200, and may output the image data that is read out to the outside of the imaging apparatus in playback.

A display unit 223 is a display composed of, for example, a thin film transistor (TFT) liquid crystal display, an organic electroluminescent (El) display, or an electronic viewfinder (EVF). The display unit 223 displays an image based on the image data temporarily stored in the internal memory 221, an image based on the image data stored in the storage medium, which is capable of being loaded in the imaging apparatus, a setup screen of the imaging apparatus 200, and so on.

An operation member 224 is composed of, for example, buttons, switches, keys, and a mode dial, which are provided on the imaging apparatus 200, or a touch panel, which is used also as the display unit 223. An instruction from a user to, for example, set a mode or instruct shooting is supplied to the system control circuit 220 through the operation member 224.

The imaging unit 211, the zoom control circuit 215, the ranging system 216, the image processing circuit 217, the AF control circuit 218, the communication unit 219, the system control circuit 220, the internal memory 221, the external memory I/F 222, the display unit 223, and the operation member 224 are connected to a common bus 225. The common bus 225 is a signal line for transmission and reception of signals between the respective blocks.

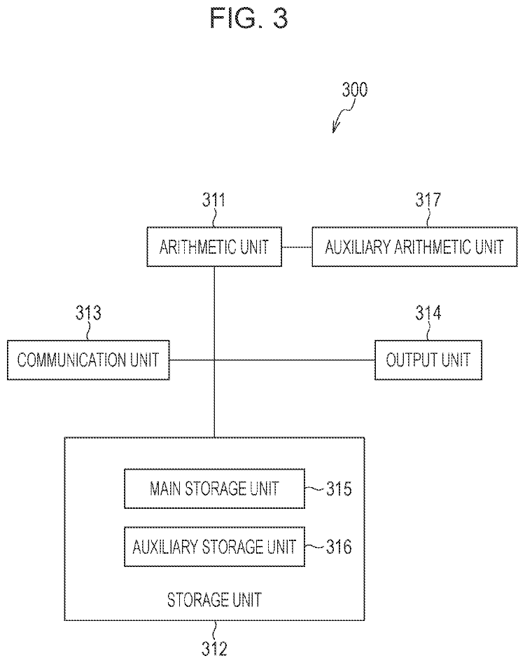

FIG. 3 is a diagram illustrating an example of the hardware configuration of the image processing apparatus 300 included in the image processing system 1. The image processing apparatus 300 includes an arithmetic unit 311 composed of a CPU, a storage unit 312, a communication unit 313, an output unit 314, and an auxiliary arithmetic unit 317. The storage unit 312 is composed of a main storage unit 315 (for example, a read only memory (ROM) or a random access memory (RAM)) and an auxiliary storage unit 316 (for example, a magnetic disk drive or a solid state drive (SSD)).

The communication unit 313 is composed as a wireless communication module for communication with an external device via the communication network. The output unit 314 outputs data processed in the arithmetic unit 311 and data stored in the storage unit 312 to a display, a printer, or an external network connected to the image processing apparatus 300.

The auxiliary arithmetic unit 317 is an integrated circuit (IC) for auxiliary arithmetic operation used under the control of the arithmetic unit 311. A graphic processing unit (GPU) may be used as an example of the auxiliary arithmetic unit. Since the GPU includes multiple product-sum operators and excels in matrix calculation although the GPU is originally a processor for image processing, the GPU is also often used as a processor that performs a signal learning process. The GPU is generally used in a deep learning process. For example, Jetson TX2 Module manufactured by NVIDIA corporation may be used as the auxiliary arithmetic unit 317. The FPGA or the ASIC may be used as the auxiliary arithmetic unit 317. The auxiliary arithmetic unit 317 extracts the affected area 102 of the subject 101 from the image data.

The arithmetic unit 311 is capable of realizing various functions including arithmetic processing for calculating the size and the length of the affected area 102 extracted by the auxiliary arithmetic unit 317 by executing programs stored in the storage unit 312. In addition, the arithmetic unit 311 controls the order in which the respective functions are performed.

The image processing apparatus 300 may include one arithmetic unit 311 and one storage unit 312 or multiple arithmetic units 311 and multiple storage units 312. In other words, the image processing apparatus 300 performs the functions described below when at least one processing unit (CPU) is connected to at least one storage unit and the at least one processing unit executes a program stored in the at least one storage unit. Instead of the CPU, the FPGA, the ASIC, or the like may be used as the arithmetic unit 311.

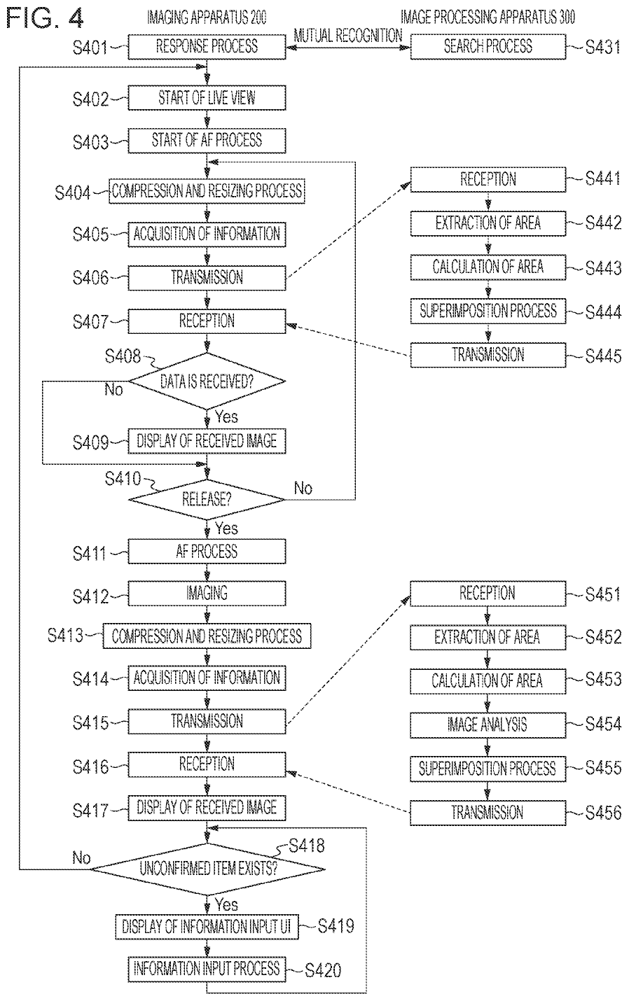

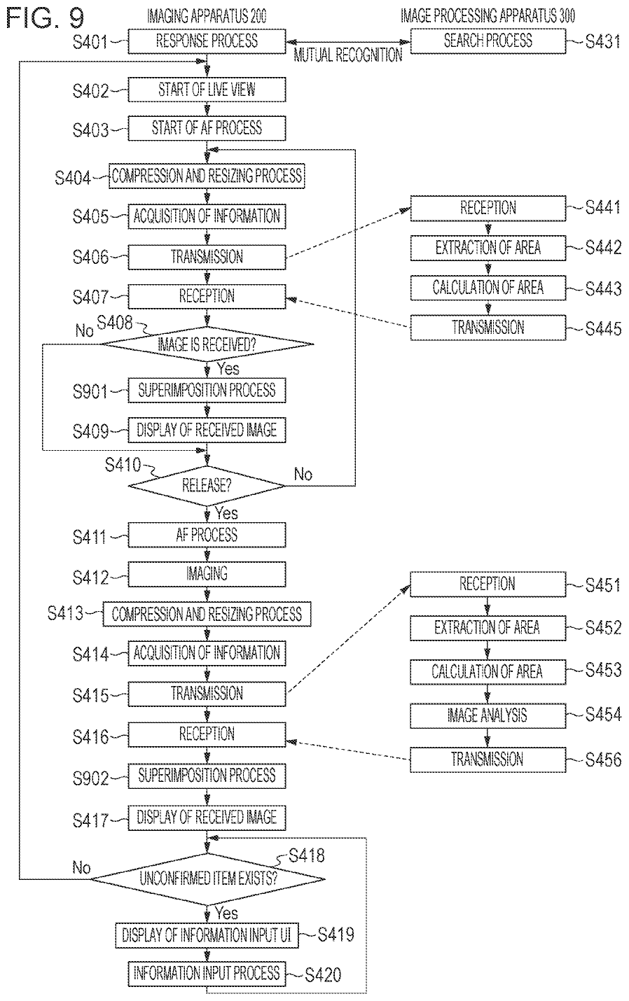

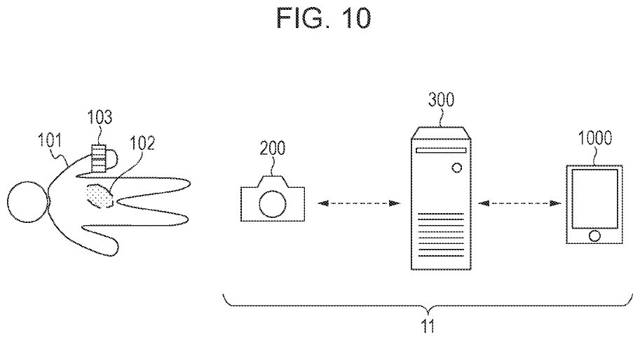

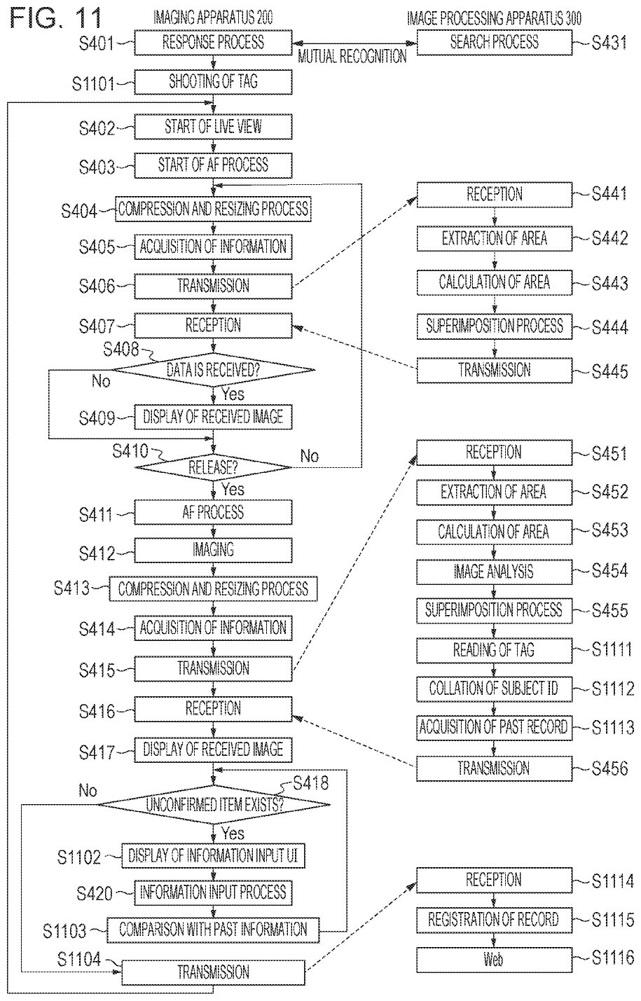

FIG. 4 is a work flow chart illustrating the operation of the image processing system 1 according to the first embodiment. Referring to FIG. 4, Step is denoted by S. In other words, Step 401 is denoted by S401. The same applies to FIG. 9, FIG. 11, and FIG. 15 described below.

In the work flow chart in FIG. 4, Step 401 to Step 420 are performed by the imaging apparatus 200 and Step 431, Step 441 to Step 445, and Step 451 to Step 456 are performed by the image processing apparatus 300.

First, the imaging apparatus 200 and the image processing apparatus 300 are connected to a network (not illustrated) conforming to the Wi-Fi standard, which is a wireless LAN standard. In Step 431, the image processing apparatus 300 performs a search process of the imaging apparatus 200 to which the image processing apparatus 300 is to be connected. In Step 401, the imaging apparatus 200 performs a response process in response to the search process. For example, Universal Plug and Play (UPnP) is used as a technology to search for a device over the network. In the UPnP, the individual apparatuses are identified using universal unique identifiers (UUIDs).

In response to connection of the imaging apparatus 200 to the image processing apparatus 300, in Step 402, the imaging apparatus 200 starts a live view process. The imaging unit 211 generates image data and the image processing circuit 217 applies a developing process necessary for generating the image data for live view display to the image data. Repeating these processes causes a live view video of a certain frame rate to be displayed in the display unit 223.

In Step 403, the ranging system 216 calculates the distance information about the subject using any of the methods described above and the AF control circuit 218 starts an AF process to drive and control the lens group 212 so that the subject is in focus. When the point of focus is adjusted using the TV-AF or the contrast AF, the distance information from the position of the focus lens in the in-focus state to the subject 101 that is in focus is calculated. The position that is to be in focus may be the subject positioned at the center of the image data or the subject existing at the position closest to the imaging apparatus 200. When the distance map of the subject is acquired, a target area may be estimated from the distance map and the focus lens may be focused on the position. Alternatively, when the position of the pressure ulcer 102 on a live view image is identified by the image processing apparatus 300, the focus lens may be focused on the position of the pressure ulcer on the live view image. The imaging apparatus 200 repeatedly performs the display of the live view video and the AF process until depression of a release button is detected in Step 410.

In Step 404, the image processing circuit 217 performs the developing process and the compression process to any image data captured for the live view to generate, for example, the image data conforming to the JPEG standard. Then, the image processing circuit 217 performs a resizing process to the image data subjected to the compression process to reduce the size of the image data.

In Step 405, the communication unit 219 acquires the image data subjected to the resizing process in Step 404 and the distance information calculated in Step 403. In addition, the communication unit 219 acquires information about the zoom magnification and information about the size (the number of pixels) of the image data subjected to the resizing process. When the imaging unit 211 has the single focus without the zoom function, it is not necessary to acquire the information about the zoom magnification.

In Step 406, the communication unit 219 transmits the image data acquired in Step 405 and at least one piece of information including the distance information to the image processing apparatus 300 through the wireless communication.

Since it takes a longer time to perform the wireless communication with the increasing size of the image data to be transmitted, the size of the image data after the resizing process in Step 405 is determined in consideration of a permitted communication time. However, since the accuracy of extraction of the affected area, which is performed by the image processing apparatus 300 in Step 433 described below, is influenced if the image data has an excessively reduced size, it is necessary to consider the accuracy of the extraction of the affected area, in addition to the communication time.

Step 404 to Step 406 may be performed for each frame or may be performed once per several frames.

The operation goes to description of the steps performed by the image processing apparatus 300.

In Step 441, the communication unit 313 in the image processing apparatus 300 receives the image data and the at least one piece of information including the distance information, which are transmitted from the communication unit 219 in the imaging apparatus 200.

In Step 442, the arithmetic unit 311 and the auxiliary arithmetic unit 317 in the image processing apparatus 300 extract the affected area 102 of the subject 101 from the image data received in Step 441. As the method of extracting the affected area 102, semantic segmentation using the deep learning is performed. Specifically, a high-performance computer for learning (not illustrated) is caused to learn a neural network model using multiple actual pressure ulcer images as teacher data in advance to generate a learned model. The auxiliary arithmetic unit 317 receives the generated learned model from the high-performance computer and estimates the area of the pressure ulcer, which is the affected area 102, from the image data based on the learned model. A fully convolutional network (FCN), which is the segmentation model using the deep learning, is applied as an example of the neural network model. Here, inference of the deep learning is processed by the auxiliary arithmetic unit 317, which excels in parallel execution of the product-sum operation. The inference process may be performed by the FPGA or the ASIC. The area segmentation may be realized using another deep learning model. The segmentation method is not limited to the deep learning and, for example, graph cut, area growth, edge detection, divide and conquer, or the like may be used as the segmentation method. In addition, learning of the neural network model using the image of the pressure ulcer as the teacher data may be performed in the auxiliary arithmetic unit 317.

In Step 443, the arithmetic unit 311 calculates the area of the affected area 102 as information indicating the size of the affected area 102 extracted by the auxiliary arithmetic unit 317.

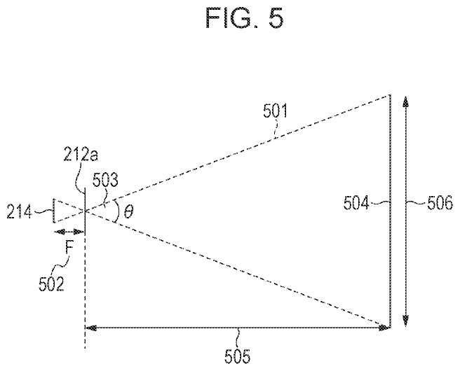

FIG. 5 is a diagram for describing how the area of the affected area 102 is calculated. The imaging apparatus 200, which is a common camera, is capable of being processed as a pin-hole model illustrated in FIG. 5. Incident light 501 passes through the principal point of a lens 212a and is received on the imaging plane of the image sensor 214. When the lens group 212 is approximated to the thin single lens 212a, the principal point at the front side is considered to coincide with the principal point at the back side. Adjusting the point of focus of the lens 212 so that an image is formed on the planar surface of the image sensor 214 enables the imaging apparatus to be focused on a subject 504. Varying a focal length 502, which is the distance from the imaging plane to the principal point of the lens, varies an angle of view 503 to vary the zoom magnification. At this time, a width 506 of the subject on the focal plane is geometrically determined from the relationship between the angle of view 503 of the imaging apparatus and a subject distance 505. The width 506 of the subject is calculated using a trigonometric function. Specifically, the width 506 of the subject is determined based on the relationship between the angle of view 503, which is varied with the focal length 502, and the subject distance 505. The value of the width 506 of the subject is divided by the number of pixels on each line of the image data to calculate the length on the focal plane corresponding to one pixel on the image data.

Accordingly, the arithmetic unit 311 calculates the area of the affected area 102 as the product of the number of pixels in the extracted area, which is acquired from the result of extraction of the affected area in Step 442, and the area of one pixel, which is acquired from the length on the focal plane corresponding to one pixel on the image. The length on the focal plane corresponding to one pixel on the image, which corresponds to the combination of the focal length 502 and the subject distance 505, may be calculated in advance to be prepared as table data. The image processing apparatus 300 may store the table data corresponding to the imaging apparatus 200 in advance.

In order to accurately calculate the area of the affected area 102 using the above method, it is assumed that the subject 504 is the planar surface and the planar surface is vertical to the optical axis. If the distance information received in Step 441 is the distance information or the distance map at multiple positions in the image data, the inclination or the variation in the depth direction of the subject may be detected to calculate the area based on the detected inclination or the variation.

In Step 444, the arithmetic unit 311 generates image data resulting from superimposition of information indicating the result of extraction of the affected area 102 and the information indicating the size of the affected area 102 on the image data used for the extraction of the affected area 102.

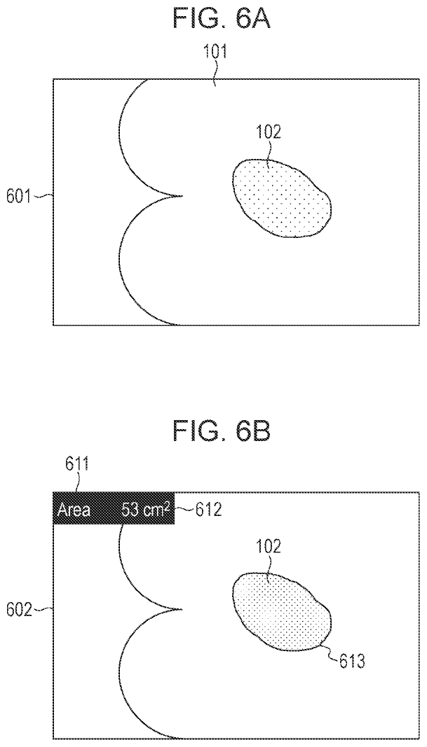

FIG. 6A and FIG. 6B are diagrams illustrating how the information indicating the result of extraction of the affected area 102 and the information indicating the size of the affected area 102 are superimposed on the image data. An image 601 in FIG. 6A is an image displayed using the image data before the superimposition process and includes the subject 101 and the affected area 102. A superimposed image 602 in FIG. 6B is an image based on the image data after the superimposition process. FIG. 6A and FIG. 6B indicate that the affected area 102 is close to the hip.

The arithmetic unit 311 superimposes a label 611 at the upper left corner of the superimposed image 602. A character string 612 indicating the area value of the affected area 102 is displayed on the label 611 with white characters on the black background as the information indicating the size of the affected area 102.

The background color and the color of the character string on the label 611 are not limited to black and white, respectively, as long as the background and the character string are easily visible. An amount of transmission may be set and α blending may be performed to the set amount of transmission to enable confirmation of the portion on which the label is superimposed.

In addition, an index 613 indicating an estimated area of the affected area 102, extracted in Step 442, is superimposed on the superimposed image 602. Performing the α blending of the index 613 indicating the estimated area and the image data on which the image 601 is based for superimposition at the position where the estimated area exists enables the user to confirm whether the estimated area on which the area of the affected area is based is appropriate. The color of the index 613 indicating the estimated area is not desirably equal to the color of the subject. The transmittance of the α blending is desirably within a range in which the estimated area is capable of being recognized and the original affected area 102 is also capable of being confirmed. Since the user is capable of confirming whether the estimated area is appropriate without the display of the label 611 when the index 613 indicating the estimated area of the affected area 102 is superimposed, Step 443 may be omitted.

In Step 445, the communication unit 313 in the image processing apparatus 300 transmits the information indicating the result of extraction of the affected area 102 that is extracted and the information indicating the size of the affected area 102 to the imaging apparatus 200. In the present embodiment, the communication unit 313 transmits the image data including the information indicating the size of the affected area 102, which is generated in Step 444, to the imaging apparatus 200 through the wireless communication.

The operation goes back to description of the steps performed by the imaging apparatus 200.

In Step 407, the communication unit 219 in the imaging apparatus 200 receives any image data that includes the information indicating the size of the affected area 102 and that is newly generated in the image processing apparatus 300.

In Step 408, the system control circuit 220 goes to Step 409 if the image data including the information indicating the size of the affected area 102 is received in Step 407 and otherwise goes to Step 410.

In Step 409, the display unit 223 displays the image data including the information indicating the size of the affected area 102, which is received in Step 407, for a certain time period. Here, the display unit 223 displays the superimposed image 602 illustrated in FIG. 6B. Superimposing the information indicating the result of extraction of the affected area 102 on the live view image in the above manner enables the user to perform the shooting after the user confirms whether the area of the affected area and the estimated area are appropriate. Although the example is described in the present embodiment in which both the index 613 indicating the estimated area of the affected area 102 and the information about the size of the affected area 102 are displayed, either of the index 613 indicating the estimated area of the affected area 102 and the information about the size of the affected area 102 may be displayed.

In Step 410, the system control circuit 220 determines whether the release button included in the operation member 224 is depressed. If the release button is not depressed, the imaging apparatus 200 goes back to Step 404. If the release button is depressed, the imaging apparatus goes to Step 411.

In Step 411, the ranging system 216 calculates the distance information about the subject and the AF control circuit 218 performs the AF process to drive and control the lens group 212 so that the subject is in focus using the same method as in Step 403. If the affected area 102 has been extracted from the live view image, the ranging system 216 calculates the distance information about the subject at the position where the affected area 102 exists.

In Step 412, the imaging apparatus 200 captures a still image.

In Step 413, the image processing circuit 217 performs the developing process and the compression process to the image data generated in Step 412 to generate, for example, the image data conforming to the JPEG standard. Then, the image processing circuit 217 performs the resizing process to the image data subjected to the compression process to reduce the size of the image data. The size of the image data subjected to the resizing process in Step 413 is equal to or greater than that of the image data subjected to the resizing process in Step 404. This is because priority is given to the accuracy of the measurement of the affected area 102. Here, the image data is resized to about 4.45 megabytes with 1,440 pixels×1,080 pixels in 4-bit RGB color. The size of the resized image data is not limited to this. Alternatively, the operation may go to the subsequent step using the generated image data conforming to the JPEG standard without the resizing process.

In Step 414, the communication unit 219 acquires the image data, which is generated in Step 413 and which is subjected to the resizing process (or which is not subjected to the resizing process), and the distance information calculated in Step 411. In addition, the communication unit 219 also acquires the information about the zoom magnification and the information about the size (the number of pixels) of the image data subjected to the resizing process. When the imaging unit 211 has the single focus without the zoom function, it is not necessary to acquire the information about the zoom magnification. When the image processing apparatus 300 has the information about the size of the image data in advance, it is not necessary to acquire the information about the image data.

In Step 415, the communication unit 219 transmits the image data acquired in Step 414 and at least one piece of information including the distance information to the image processing apparatus 300 through the wireless communication.

The operation goes to description of the steps performed by the image processing apparatus 300.

In Step 451, the communication unit 313 in the image processing apparatus 300 receives the image data and the at least one piece of information including the distance information, which are transmitted from the communication unit 219 in the imaging apparatus 200.

In Step 452, the arithmetic unit 311 and the auxiliary arithmetic unit 317 in the image processing apparatus 300 extract the affected area 102 of the subject 101 from the image data received in Step 441. Since the details of the step is the same as in Step 442, the detailed description of Step 452 is omitted herein.

In Step 453, the arithmetic unit 311 calculates the area of the affected area 102 as an example of the information indicating the size of the affected area 102 extracted by the auxiliary arithmetic unit 317. Since the details of the step is the same as in Step 443, the detailed description of Step 453 is omitted herein.

In Step 454, the arithmetic unit 311 performs image analysis to calculate the major axis length and the minor axis length of the extracted affected area and the area of a rectangle circumscribed around the affected area based on the length on the focal plane corresponding to one pixel on the image, calculated in Step 453. The DESIGN-R (registered trademark), which is the evaluation index of the pressure ulcer, defines that the size of the pressure ulcer is calculated by measuring the value of the product of the major axis length and the minor axis length. In the image processing system of the present invention, the analysis of the major axis length and the minor axis length enables the compatibility with the data that has been measured in the DESIGN-R (registered trademark) to be ensured. Since the strict definition is not provided in the DESIGN-R (registered trademark), multiple mathematical methods of calculating the major axis length and the minor axis length are considered.

As one example of the method of calculating the major axis length and the minor axis length, first, the arithmetic unit 311 calculates a minimum bounding rectangle, which is a rectangle having the minimum area, among the rectangles circumscribed around the affected area 102. Then, the arithmetic unit 311 calculates the lengths of the long side and the short side of the rectangle. The length of the long side is calculated as the major axis length and the length of the short side is calculated as the minor axis length. Then, the arithmetic unit 311 calculates the area of the rectangle based on the length on the focal plane corresponding to one pixel on the image, calculated in Step 453.

As another example of the method of calculating the major axis length and the minor axis length, a maximum Feret diameter, which is the maximum caliper length, may be selected as the major axis length and a minimum Feret diameter may be selected as the minor axis length. Alternatively, the maximum Feret diameter, which is the maximum caliper length, may be selected as the major axis length and a length measured in a direction orthogonal to the axis of the maximum Feret diameter may be selected as the minor axis length. The method of calculating the major axis length and the minor axis length may be arbitrarily selected based on the compatibility with the result of measurement in the related art.

The calculation of the major axis length and the minor axis length of the affected area 102 and the area of the rectangle is not performed to the image data received in Step 441. Since the confirmation of the result of extraction of the affected area 102 by the user is intended during the live view, the step of the image analysis in Step 454 is omitted to reduce the processing time.

Step 454 may be omitted when the acquisition of the information about the actual area of the pressure ulcer is intended without the evaluation of the size based on the DESIGN-R (registered trademark). In this case, it is assumed in the subsequent steps that the information about the size, which is the evaluation item in the DESIGN-R (registered trademark), does not exist.

In Step 455, the arithmetic unit 311 generates image data resulting from superimposition of the information indicating the result of extraction of the affected area 102 and the information indicating the size of the affected area 102 on the image data used as the target of the extraction of the affected area 102.

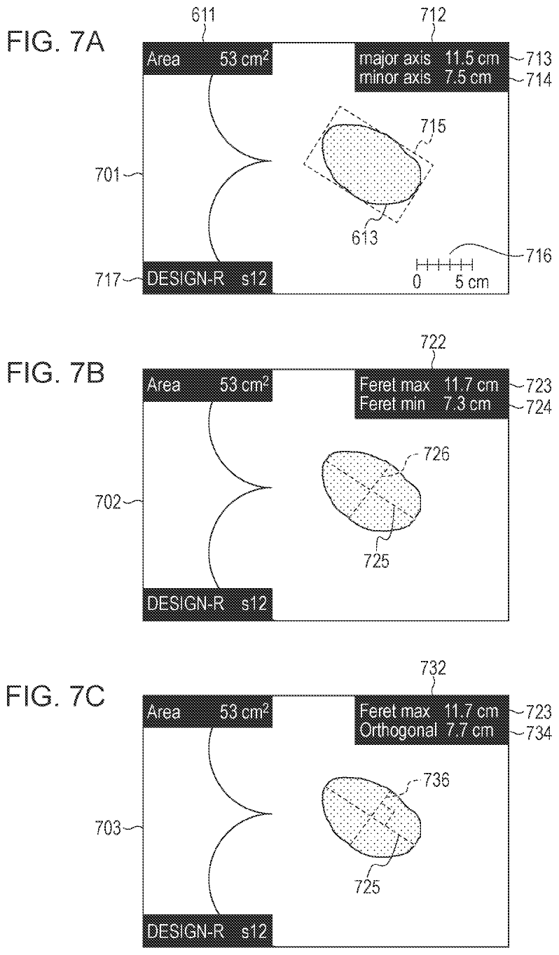

FIG. 7A to FIG. 7C are diagrams for describing the method of superimposing the information indicating the result of extraction of the affected area 102 and the information indicating the size of the affected area, which includes the major axis length and the minor axis length of the affected area 102, on the image data. Since multiple pieces of information indicating the size of the affected area 102 are considered, a superimposed image 701 in FIG. 7A, a superimposed image 702 in FIG. 7B, and a superimposed image 703 in FIG. 7C are separately described.

In the case of the superimposed image 701 in FIG. 7A, the minimum bounding rectangle is used as the method of calculating the major axis length and the minor axis length. The label 611 is superimposed at the upper left corner of the superimposed image 701. The character string 612 indicating the area value of the affected area 102 is displayed on the label 611 with white characters on the black background as the information indicating the size of the affected area 102, as in FIG. 6B. In addition, a label 712 is superimposed at the upper right corner of the superimposed image 701. The major axis length and the minor axis length calculated based on the minimum bounding rectangle are displayed on the label 712 as the information indicating the size of the affected area 102. A character string 713 indicates the major axis length (cm) and a character string 714 indicates the minor axis length (cm). A rectangular frame 715 representing the minimum bounding rectangle is displayed around the affected area 102 on the superimposed image 701. Superimposing the rectangular frame 715 with the major axis length and the minor axis length enables the user to confirm the place for which the length is being measured in the image.

In addition, a scale bar 716 is superimposed at the lower right corner of the superimposed image 701. The scale bar 716 is used for measuring the size of the affected area 102 and the size of the scale bar on the image data is varied with the distance information. Specifically, the scale bar 716 is a bar on which scale marks from 0 cm to 5 cm are indicated in units of 1 cm based on the length on the focal plane corresponding to one pixel on the image, calculated in Step 453, and is matched with the size on the focal plane of the imaging apparatus, that is, on the subject. The user is capable of knowing the approximate size of the subject or the affected area with reference to the scale bar.

Furthermore, an evaluation value of Size in the DESIGN-R (registered trademark) described above is superimposed at the lower left corner of the superimposed image 701. The evaluation value of Size in the DESIGN-R (registered trademark) is classified into the seven stages described above based on the value given by measuring the major axis length (cm) and the minor axis length (the maximum diameter orthogonal to the major axis length) (cm) of the skin injury range and multiplying the major axis length by the minor axis length. In the present embodiment, the evaluation value resulting from replacement of the major axis length and the minor axis length with the value that is output using the calculation methods is superimposed.

In the case of the superimposed image 702 in FIG. 7B, the maximum Feret diameter is used as the major axis length and the minimum Feret diameter is used as the minor axis length. A label 722 is superimposed at the upper right corner of the superimposed image 702. A major axis length character string 723 and a minor axis length character string 724 are displayed on the label 722. In addition, an auxiliary line 725 corresponding to the measurement position of the maximum Feret diameter and an auxiliary line 726 corresponding to the minimum Feret diameter are displayed in the affected area 102 on the superimposed image 702. Superimposing the auxiliary lines with the major axis length and the minor axis length enables the user to confirm the place for which the length is being measured in the image.

The superimposed image 703 in FIG. 7C is the same as the superimposed image 702 in the major axis length. However, the minor axis length is measured not as the minimum Feret diameter but as the length measured in the direction orthogonal to the axis of the maximum Feret diameter on the superimposed image 703. A label 732 is superimposed at the upper right corner of the superimposed image 703. The major axis length character string 723 and a minor axis length character string 734 are displayed on the label 732. In addition, the auxiliary line 725 corresponding to the measurement position of the maximum Feret diameter and an auxiliary line 736 corresponding to the length measured in the direction orthogonal to the axis of the maximum Feret diameter are displayed in the affected area 102 on the superimposed image 703.

Any one of the pieces of information to be superimposed on the image data, illustrated in FIG. 7A to FIG. 7C, may be used or a combination of multiple pieces of the information may be used. Alternatively, the user may be capable of selecting the information to be displayed. The superimposed images illustrated in FIG. 6B and FIG. 7A to FIG. 7C are only examples and the display mode, the display positions, the font type, the font size, the font color, the positional relationship, and so on of the affected area 102 and the information indicating the size of the affected area 102 may be varied depending on various conditions.

In Step 456, the communication unit 313 in the image processing apparatus 300 transmits the information indicating the result of extraction of the affected area 102 that is extracted and the information indicating the size of the affected area 102 to the imaging apparatus 200. In the present embodiment, the communication unit 313 transmits the image data including the information indicating the size of the affected area 102, which is generated in Step 455, to the imaging apparatus 200 through the wireless communication.

The operation goes back to description of the steps performed by the imaging apparatus 200.

In Step 416, the communication unit 219 in the imaging apparatus 200 receives the image data including the information indicating the size of the affected area 102, which is generated in the image processing apparatus 300.

In Step 417, the display unit 223 displays the image data including the information indicating the size of the affected area 102, which is received in Step 416, for a certain time period. Here, the display unit 223 displays any of the superimposed images 701 to 703 illustrated in FIG. 7A to FIG. 7C, respectively, and the operation goes to Step 418 after the certain time period elapsed.

In Step 418, it is determined whether affected area information for which no value is input exists. The affected area information indicates information indicating the region of the affected area and the evaluation value of each evaluation item in the DESIGN-R (registered trademark) described above. The evaluation value of the evaluation item concerning Size is automatically input based on the information indicating the size, which is received in Step 416.

If the affected area information for which no value is input exists in Step 418, the operation goes to Step 419. If all the affected area information is input in Step 418, the operation goes back to Step 402 to start the live view again.

In Step 419, the system control circuit 220 displays a user interface prompting the user to input the affected area information in the display unit 223.

In Step 420, upon input of the affected area information by the user, the operation goes back to Step 418.

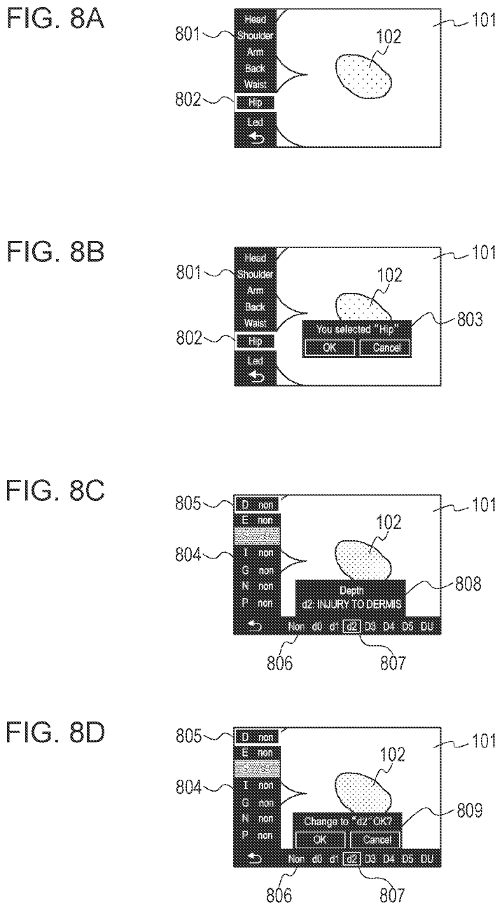

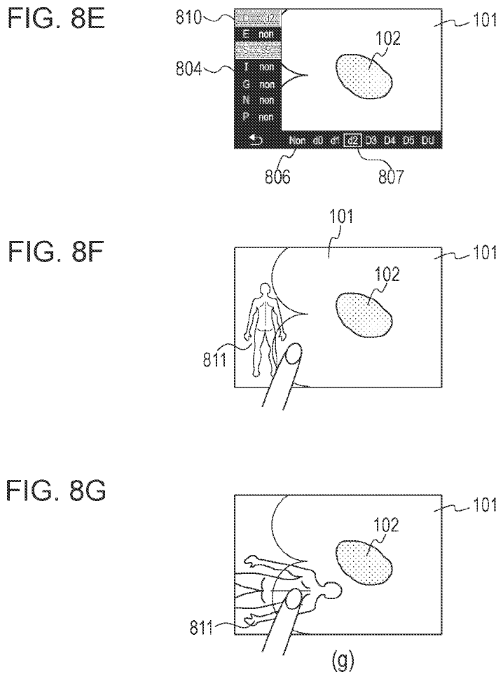

FIG. 8A to FIG. 8G are diagrams for describing how to cause the user to input the affected area information in Step 419 and Step 420.

FIG. 8A is a screen prompting the user to input the region of the affected area in the affected area information.

Region selection items 801 for specifying the regions: Head, Shoulder, Arm, Back, Waist, Hip, and Leg of the affected area are displayed in the display unit 223. An item for completing the input of the affected area information is provided below the region selection items 801. Selecting the item enables the input of the affected area information to be terminated even if part of the affected area information is not input.

The user is capable of specifying the region in which the affected area that is shot exists with the operation member 224. The item selected by the user is displayed with being surrounded by a frame line 802. The state in which Hip is selected is displayed in FIG. 8A. Since two or more affected areas may exist in one region selected from the region selection items 801, selection of multiple items, such as Hip1, Hip2, and Hip3, may further be available.

FIG. 8B is a screen causing the user to confirm whether the selected region is appropriate after the region including the affected area is selected in FIG. 8A. Upon confirmation of the selected region with a user's operation, the display unit 223 displays a screen illustrated in FIG. 8C.

FIG. 8C is a screen prompting the user to input the evaluation value of each evaluation item in the DESIGN-R (registered trademark) in the affected area information.

An evaluation item selection portion 804 is displayed on the left side of the screen. The respective items: D (Depth), E (Exudate), S (Size), I (Inflammation/Infection), G (Granulation), N (Necrotic tissue), and P (Pocket) and information indicating whether each item is input are displayed with the image of the affected area. In FIG. 8C, an evaluation value “s9” is displayed for S (Size), which has been analyzed from the image, and “non” indicating that the item has not been confirmed is displayed for the remaining evaluation items. Hatching of the item S (Size) indicates that the item S (Size) has been input.

The user is capable of specifying the evaluation item with the operation member 224. The selected evaluation item (D (Depth) here) is displayed with being surrounded by a frame line 805.

The evaluation values of a severity level of the evaluation item selected on the left side of the screen are superimposed on the bottom of the screen as a severity level selection portion 806. In FIG. 8C, d0, d1, d2, D3, D4, D5, and DU, which are the evaluation values indicating the severity level of D (Depth), are displayed.

The user is capable of selecting any of the evaluation values with the operation member 224. The selected evaluation value is displayed with being surrounded by a frame line 807, and a descriptive text 808 (description of the evaluation item Depth and the severity level d2: injury to dermis) of the evaluation value is also displayed. The evaluation value may be input by the user who inputs a character string.

FIG. 8D illustrates a confirmation notification 809 for inquiring of the user whether the selected evaluation value is appropriate after the evaluation value is selected in FIG. 8C.

Upon confirmation that there is no problem about the selected evaluation value by the user with the operation member 224, the screen makes a transition to a screen illustrated in FIG. 8E.

In FIG. 8E, the display of an evaluation item 810 of D (Depth) is changed from “non” to “d2” in response to the input of the evaluation value and the evaluation item 810 is hatched.

Similarly, screens prompting the user to input the evaluation values for E (Exudate), I (Inflammation/Infection), G (Granulation), N (Necrotic tissue), and P (Pocket) are displayed until the evaluation values are input for all the evaluation items.

In response to input of the evaluation values of all the evaluation items, the user is notified of completion of the input of the affected area information. Then, the operation goes back to Step 402 to start the live view process.

As described above, in the first embodiment, the function is provided to cause the user to input the affected area information by prompting the user to input the evaluation value of the evaluation item that is not subjected to the automatic analysis and the information about the region of the affected area after the affected area is shot in Step 418 to Step 420. It is possible to input the affected area information, which is input using another medium in the related art, only with the imaging apparatus in the above manner.

In addition, it is possible to prevent input omission of the affected area information by determining whether all the pieces of affected area information are input and sequentially prompting the user to input the evaluation items that are not input before the next affected area is shot.

Voice recognition input may be used as the operation member 224 according to the first embodiment.

In FIG. 8A, the regions are displayed using the characters, such as “Head” and “Shoulder”, when the region of the affected area is input and the characters are selected. In contrast, as illustrated in FIG. 8F, a configuration may be adopted in which a human body model 811 is displayed in the display unit 223 and the user is caused to specify the region of the affected area using a touch sensor provided on the display unit 223.

In addition, as illustrated in FIG. 8G, a configuration may be adopted in which the human body model 811 is enlarged, reduced, or rotated to enable the region of the affected area to be easily selected.

Although the hatching is used for indicating that the input of the evaluation value is completed for the evaluation item in FIG. 8E, the luminance of the characters may be reduced or the characters may be highlighted. Other display methods may be used as long as the fact that the evaluation value has been input for the evaluation item is explicitly indicated to the user.

Although the DESIGN-R (registered trademark) is used as the available evaluation index of the pressure ulcer in the present example, the evaluation index is not limited to this. Another evaluation index, such as Bates-Jensen Wound Assessment Tool (BWAT), Pressure Ulcer Scale for Healing (PUSH), or Pressure Sore Status Tool (PSST), may be used. Specifically, a user interface used for inputting the evaluation items in the BWAT, the PUSH, the PSST, or the like may be displayed in response to the acquisition of the result of extraction of the area of the pressure ulcer and the information about the size of the extracted area.

Although the example of the configuration intended to input of the evaluation values of the evaluation items of the pressure ulcer is described in the present example, the input of the evaluation values of the evaluation items in another skin disease may be intended as long as the visual evaluation items are used. For example, Severity Scoring of Atopic Dermatitis (SCORAD) in atopic dermatitis and Body Surface Area, Psoriasis Area and Severity Index (PASI) in psoriasis are exemplified.

As described above, according to the present embodiment, the image processing system is provided in which the information indicating the size of the affected area is displayed in the display unit 223 in the imaging apparatus 200 in response to shooting of the affected area 102 by the user with the imaging apparatus 200. Accordingly, it is possible to reduce the burden on the medical personnel in the evaluation of the size of the affected area of the pressure ulcer and the burden on the patient to be evaluated. In addition, the calculation of the size of the affected area based on a program enables the individual difference to be reduced, compared with the case in which the medical personnel manually measures the size of the affected area, to improve the accuracy in the evaluation of the size of the pressure ulcer. Furthermore, it is possible to calculate the area of the affected area, which is the evaluation value, and display the calculated area of the affected area in order to indicate the size of the pressure ulcer more accurately.

Since the function to confirm whether the estimated area of the affected area is appropriate by the user in the live view display is not essential, a configuration may be adopted in which Step 406, Step 407, and Step 441 to Step 445 are omitted.

The image processing apparatus 300 may store the information indicating the result of extraction of the affected area 102, the information indicating the size of the affected area 102, and the image data about the superimposed image on which the information indicating the result of extraction of the affected area 102 and the information indicating the size of the affected area 102 are superimposed in the storage unit 312. The output unit 314 is capable of outputting at least one piece of information stored in the storage unit 312 or the image data to an output device, such as a display connected to the image processing apparatus 300. The display of the superimposed image in the display enables another user different from the user who shots the affected area 102 to acquire the image of the affected area 102 in real time or acquire the image of the affected area 102 which has been captured and the information indicating the size of the affected area 102. The arithmetic unit 311 in the image processing apparatus 300 may have a function to display a scale bar or the like which arbitrarily varies the position and the angle for the image data to be transmitted from the output unit 314 to the display. The display of such a scale bar enables the user who watches the display to measure the length of an arbitrary place of the affected area 102. The width of the scale of the scale bar is desirably adjusted automatically based on the distance information received in Step 451, the information about the zoom magnification, the information about the size (the number of pixels) of the image data subjected to the resizing process, and so on.

Use of the image processing apparatus 300 in a state in which power is constantly supplied at the stationary side enables the image of the affected area 102 and the information indicating the size of the affected area 102 to be acquired at arbitrary timing with no risk of battery exhaustion. In addition, since the image processing apparatus 300, which is generally the stationary device, has high storage capacity, the image processing apparatus 300 is capable of storing a large amount of image data.

In addition, according to the present embodiment, the user is capable of inputting and recording information about the affected area 102, which is different from the information acquired from the image analysis of the image, when the user shots the affected area 102 with the imaging apparatus 200. Accordingly, it is not necessary for the user to subsequently input the evaluation of the affected area on an electronic health record or a paper medium while the user is watching the image data that is captured. Furthermore, presentation of the item that is not input to the user inhibits the user from forgetting the input of the information when the user shots the affected area.

### Second Embodiment

In the image processing system according to the first embodiment, the image processing apparatus 300 performs the process to superimpose the information indicating the result of extraction of the affected area and the information indicating the size of the affected area on the image data. In contrast, in the image processing system according to a second embodiment, the image processing circuit 217 in the imaging apparatus 200 performs the process to superimpose the information indicating the result of extraction of the affected area and the information indicating the size of the affected area on the image data.

FIG. 9 is a work flow chart illustrating the operation of the image processing system 1 according to the second embodiment.

In the work flow in FIG. 9, the superimposition process in Step 444 and Step 455 by the image processing apparatus 300 in the work flow illustrated in FIG. 4 is not performed and, instead of the superimposition process in Step 444 and Step 455 by the image processing apparatus 300, the superimposition process in Step 901 and Step 902 by the imaging apparatus 200 is added. The same processing as in the corresponding steps in FIG. 4 is performed in the steps to which the same numbers as those of the steps in FIG. 4 are given, among the steps described in FIG. 9.

In the present embodiment, the data to be transmitted from the image processing apparatus 300 to the imaging apparatus 200 in Step 445 and Step 456 for the generation of the superimposed image by the imaging apparatus 200 may not be the image data using a color scale. Since the image processing apparatus 300 does not transmit the image data but transmits metadata indicating the size of an estimated affected area and data indicating the position of the affected area, it is possible to reduce the communication traffic to increase the communication speed. The data indicating the position of the estimated affected area is data in a vector format having a smaller size. The data indicating the position of the estimated affected area may be data in a binary raster format.

Upon reception of the metadata indicating the size of the estimated affected area and the data indicating the position of the affected area from the image processing apparatus 300 in Step 407 or Step 416, the imaging apparatus 200 generates the superimposed image in Step 901 or Step 902, respectively.

Specifically, in Step 901, the image processing circuit 217 in the imaging apparatus 200 generates the superimposed image using the method described in Step 444 in FIG. 4. The image data on which the information indicating the size and the position of the estimated affected area is to be superimposed may be the image data transmitted from the imaging apparatus 200 to the image processing apparatus 300 in Step 406 or may the image data about the last frame displayed as the live view image.

In Step 902, the image processing circuit 217 in the imaging apparatus 200 generates the superimposed image using the method described in Step 455 in FIG. 4. The image data on which the information indicating the size and the position of the estimated affected area is to be superimposed is the image data transmitted from the imaging apparatus 200 to the image processing apparatus 300 in Step 415.

As described above, according to the present embodiment, since the amount of data to be transmitted from the image processing apparatus 300 to the imaging apparatus 200 is reduced, compared with the first embodiment, it is possible to reduce the communication traffic between the imaging apparatus 200 and the image processing apparatus 300 to increase the communication speed.

### Third Embodiment

FIG. 10 is a diagram schematically illustrating an image processing system 11 according to a third embodiment. The image processing system 11 illustrated in FIG. 10 includes a terminal apparatus 1000, which is an electronic device capable of Web access, in addition to the imaging apparatus 200 and the image processing apparatus 300 described above in the first and second embodiments. The terminal apparatus 1000 is composed of, for example, a tablet terminal and has a Web browser function. The terminal apparatus 1000 is capable of accessing a Web server and displaying a Hyper Text Markup Language (HTML) file that is acquired. The terminal apparatus 1000 is not limited to the tablet terminal and may be a Web browser or a device capable of displaying an image with dedicated application software. The terminal apparatus 1000 may be, for example, a smartphone or a personal computer. Although the imaging apparatus 200 and the terminal apparatus 1000 are described as separate apparatuses here, a single apparatus may be used as the imaging apparatus 200 and the terminal apparatus 1000. When the terminal apparatus 1000 is a smartphone or a tablet terminal with a camera function, the terminal apparatus 1000 is capable of serving as the imaging apparatus 200.

The arithmetic unit 311 in the image processing apparatus 300 performs a process to identify the subject from the image data, in addition to the processes described above in the first and second embodiments. In addition, the arithmetic unit 311 performs a process to store the information about the size and the position of the estimated affected area and the image data about the affected area in the storage unit 312 for each subject that is identified. The terminal apparatus 1000 is capable of causing the user to confirm the information indicating the size of the estimated affected area, which is associated with the subject, and the image data about the affected area, which are stored in the storage unit 312 in the image processing apparatus 300, using a Web browser or dedicated application software. It is assumed here for description that the terminal apparatus 1000 causes the user to confirm the image data using the Web browser.

Although the function to identify the subject from the image data, the function to store the information about the affected area or the image data for each subject that is identified, or the function to perform a Web service is performed by the image processing apparatus 300 in the present embodiment, the functions are not limitedly performed by the image processing apparatus 300. Part or all of the functions may be realized by a computer on a network different from that of the image processing apparatus 300.

Referring to FIG. 10, the subject 101 wears a barcode tag 103 as information identifying the subject. The image data about the affected area 102 that is shot is capable of being associated with an identifier (ID) of the subject, which is indicated by the barcode tag 103. The tag identifying the subject is not limited to the barcode tag and may be a two-dimensional code, such as a QR code (registered trademark), or a numerical value. Alternatively, a tag having a text described thereon may be used as the tag identifying the subject and the tag may be read using an optical character recognition (OCR) reader function installed in the image processing apparatus 300.

The arithmetic unit 311 in the image processing apparatus 300 collates the ID resulting from analysis of the barcode tag included in the image data that is captured with a subject ID registered in the storage unit 312 in advance to acquire the name of the subject 101. A configuration may be adopted in which the imaging apparatus 200 analyzes the ID and the ID is transmitted to the image processing apparatus 300.

The arithmetic unit 311 creates a record based on the image data about the affected area 102, the information indicating the size of the affected area 102 of the subject, the subject ID, the acquired name of the subject, the shooting date and time, and so on and registers the record in a database in the storage unit 312.

In addition, the arithmetic unit 311 returns the information registered in the database in the storage unit 312 in response to a request from the terminal apparatus 1000.

FIG. 11 is a work flow chart illustrating the operation of the image processing system 11 according to the third embodiment. The same processing as in the corresponding steps in FIG. 4 is performed in the steps to which the same numbers as those of the steps in FIG. 4 are given, among the steps described in FIG. 11.

Referring to FIG. 11, upon connection between the imaging apparatus 200 and the image processing apparatus 300, in Step 1101, the imaging apparatus 200 displays an instruction to cause the user to shoot the barcode tag 103 in the display unit 223 and shots the barcode tag 103 in response to the release operation by the user. Then, the operation goes to Step 402. Information about a patient ID for identifying the patient is included in the barcode tag 103. Shooting the affected area 102 after shooting the barcode tag 103 enables the shooting order to be managed based on the shooting date and time or the like to identify the image from the image of one barcode tag before the image of the next barcode tag as the image of the same subject using the subject ID. An order may be adopted in which the barcode tag 103 is shot after the affected area 102 is shot.

After the system control circuit 220 detects depression of the release button in Step 410 and Step 411 to Step 414 are performed, in Step 415, the communication unit 219 transmits the image data and at least one piece of information including the distance information to the image processing apparatus 300 through the wireless communication. The image data generated by shotting the barcode tag 103 in Step 1101 is included in the image data transmitted in Step 415, in addition to the image data generated by shooting the affected area 102.

In Step 455, the image processing apparatus 300 generates the image data about the superimposed image. Then, the operation goes to Step 1111.

In Step 1111, the arithmetic unit 311 performs a process to read a one-dimensional barcode (not illustrated) included in the image data about the barcode tag 103 shot in Step 1101 to read the subject ID identifying the subject.

In Step 1112, the subject ID that is read is collated with the subject ID registered in the storage unit 312.

In Step 1113, if the collation of the subject ID succeeded, the name of the patient registered in the database in the storage unit 312 and the past affected area information are acquired. The affected area information that is stored last is acquired here.

In Step 456, the communication unit 313 in the image processing apparatus 300 transmits the information indicating the result of extraction of the affected area 102 that is extracted, the information indicating the size of the affected area 102, and the past affected area information acquired from the storage unit 312 to the imaging apparatus 200.

In Step 416, the communication unit 219 in the imaging apparatus 200 receives the image data and the affected area information, which are transmitted from the image processing apparatus 300.

In Step 417, the display unit 223 displays the image data including the information indicating the size of the affected area 102, which is received in Step 416, for a certain time period.

In Step 418, it is determined whether the affected area information for which no value is input exists.

If the affected area information for which no value is input exists in Step 418, the operation goes to Step 1102. If all the affected area information is input in Step 418, the operation goes to Step 1104.

In Step 1102, the system control circuit 220 displays a user interface prompting the user to input the affected area information in the display unit 223 using the past affected area information.

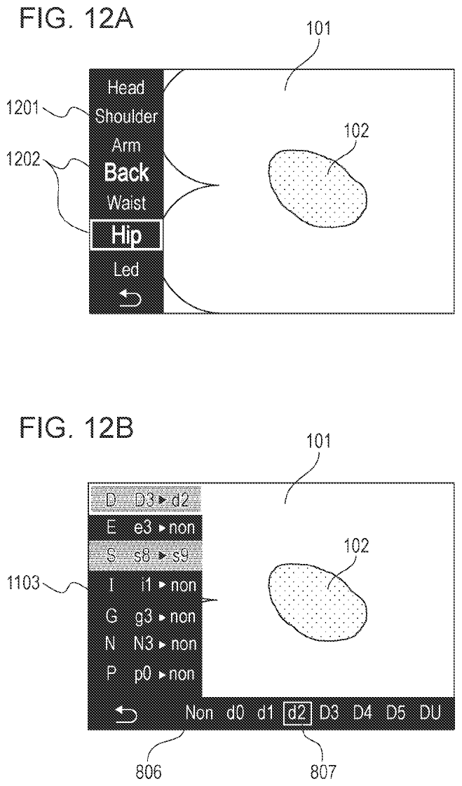

FIG. 12A and FIG. 12B are diagrams for describing how to display the affected area information that has been acquired. In FIG. 12A, the character sizes of items 1202 displayed in a region selection item 1201 on the left side of the screen are made larger for the regions for which the evaluation values of the evaluation items have been input. FIG. 12A indicates that the evaluation values of the evaluation items of the affected areas have been input for Back and Hip.

Upon input of the affected area information by the user in Step 420, in Step 1103, comparison with the past evaluation value of the evaluation item is performed to display a result of determination of whether the symptom is relieved or made worse.

In FIG. 12B, an evaluation item selection portion 1103 is displayed in three columns. The evaluation item names, the past evaluation values, and the current evaluation values are sequentially displayed from the left.

Here, the past evaluation values are compared with the current evaluation values. The green evaluation value is displayed for the item the symptom of which is determined to be relieved, and the red evaluation value is displayed for the item the symptom of which is determined to be made worse.

Upon input of the evaluation values of all the evaluation items, the user is notified of completion of the input of the affected area information. Then, the operation goes to Step 1104.

In Step 1104, the affected area information in which the evaluation values of the series of evaluation items are input and the image data are transmitted to the image processing apparatus 300 through the wireless communication. Then, the operation goes back to Step 402.

In Step 1114, the image processing apparatus 300 receives the affected area information and the image data, which are transmitted from the imaging apparatus 200.

In Step 1115, the arithmetic unit 311 creates a record based on the image data resulting from shooting of the affected area, the information about the region of the affected area 102, the evaluation value of each evaluation item of the affected area 102, the subject ID, the acquired name of the subject, the shooting date and time, and so on. In addition, the arithmetic unit 311 registers the created record in the database in the storage unit 312.

In Step 1116, the arithmetic unit 311 transmits the information registered in the database in the storage unit 312 to the terminal apparatus 1000 in response to a request from the terminal apparatus 1000.

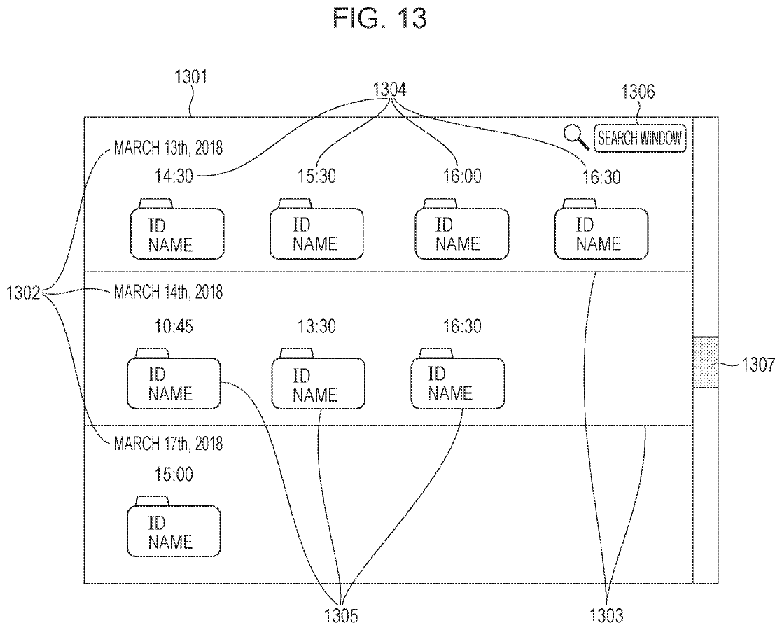

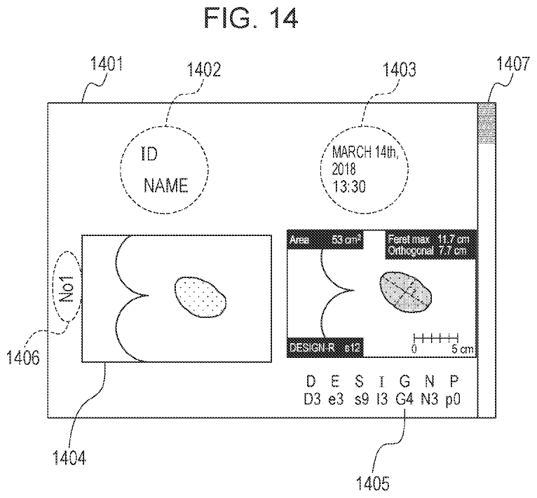

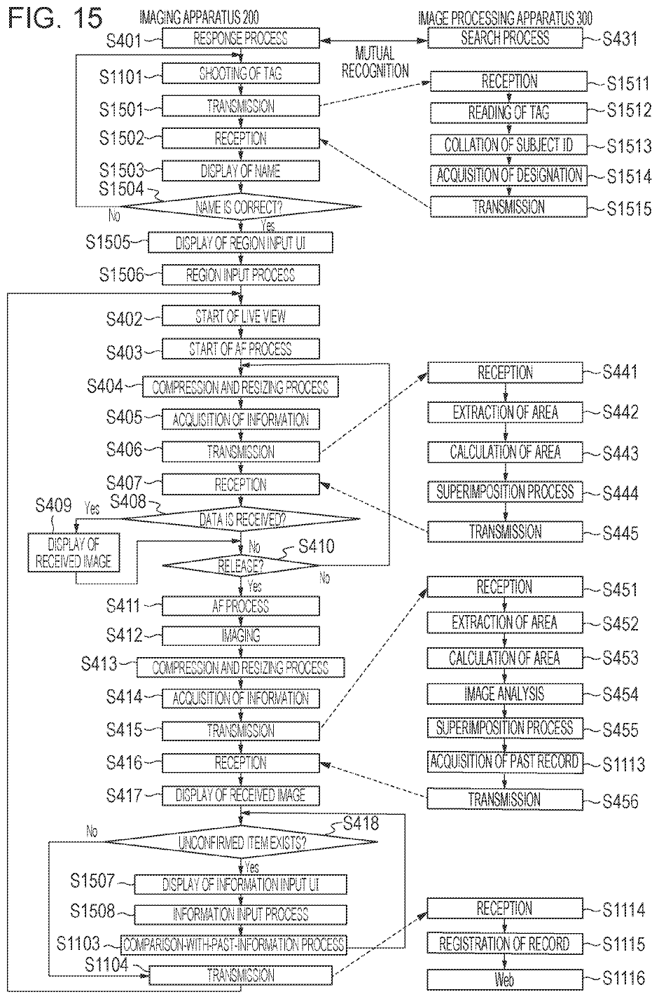

Examples of display of a browser of the terminal apparatus 1000 are described with reference to FIG. 13 and FIG. 14.

FIG. 13 is a diagram for describing an example of a data selection window displayed in the browser of the terminal apparatus 1000. A data selection window 1301 is separated using separation lines 1303 for the respective dates 1302. Icons 1305 are displayed for the respective shooting times 1304 in the area of each date. The subject ID and the name of the subject are displayed in each icon 1305 and the icon 1305 represents a data set of the same subject shot at the same time zone. A search window 1306 is provided on the data selection window 1301. Inputting the date, the subject ID, or the name of the subject in the search window 1306 enables search for the data set. In addition, operating a scroll bar 1307 enables multiple pieces of data to be displayed in an enlarged manner in a limited display area. Upon selection and clicking of the icon 1305 by the user, the browser makes a transition to a data browsing window and the user of the browser of the terminal apparatus 1000 is capable of browsing the image of the data set and the information indicating the size of the subject. In other words, a request indicating the subject and the date and time, which are specified in the terminal apparatus 1000, is transmitted from the terminal apparatus 1000 to the image processing apparatus 300. The image processing apparatus 300 transmits the image data corresponding to the request and the information indicating the size of the subject to the terminal apparatus 1000.

FIG. 14 is a diagram for describing an example of the data browsing window displayed in the browser of the terminal apparatus 1000. A subject-ID and name-of-subject 1402 of the data set selected on the data selection window 1301 and a shooting-date-and-time 1403 are displayed on a data browsing window 1401. In addition, an image 1404 based on the image data and data 1405 based on the affected area information in the image 1404 are displayed for every shooting. Furthermore, the shooting number when the affected area of the same subject is continuously shot multiple times is displayed in a number 1406. Moving a slider 1407 on the right edge of the window enables the data based on the image data and the affected area information on another shooting date and time about the same subject ID to be displayed. In addition, changing the settings enables the data based on the affected area information on multiple shooting dates and times to be displayed to facilitate comparison of the change in the symptom of the affected area.

Although the process is performed in FIG. 14 in which, after the affected area is shot, the user is caused to perform the collation of the subject ID and the selection of the region where the affected area exists, the collation of the subject ID and the selection of the region where the affected area exists may be performed before the affected area is shot.

FIG. 15 is a work flow chart illustrating a modification of the operation of the image processing system 11 according to the third embodiment. The same processing as in the corresponding steps in FIG. 11 is performed in the steps to which the same numbers as those of the steps in FIG. 11 are given, among the steps described in FIG. 15.

Upon shooting of the barcode tag 103 in Step 1101, in Step 1501, the communication unit 219 transmits the image data generated by shooting the barcode tag 103 to the image processing apparatus 300.

In Step 1511, the communication unit 313 in the image processing apparatus receives the image data generated by shooting the barcode tag 103, which is transmitted from the imaging apparatus 200.

In Step 1512, the arithmetic unit 311 performs a process to read a one-dimensional barcode included in the image data about the barcode tag 103 that is received to read the subject ID identifying the subject.

In Step 1513, the subject ID that is read is collated with the subject ID registered in the storage unit 312.

In Step 1514, if the collation of the subject ID succeeded, the name of the patient registered in the database in the storage unit 312 is acquired. If the collation failed, information indicating that the collation failed is acquired, instead of the name of the patient.

In Step 1515, the communication unit 313 in the image processing apparatus transmits the name of the patient or the information indicating that the collation of the subject ID failed to the imaging apparatus 200.

In Step 1502, the communication unit 219 in the imaging apparatus 200 receives the name of the patient, which is transmitted from the image processing apparatus 300.

In Step 1503, the system control circuit 220 displays the name of the patient in the display unit 223.

In Step 1504, the system control circuit 220 displays the name of the patient in the display unit 223. Here, the user may be caused to input the result of confirmation of whether the name of the patient is correct. If the name of the patient is not correct or if the collation of the name of the patient failed, the operation may go back to Step 1101. Displaying the name of the patient before the image of the affected area is captured prevents wrong association between the image data about the affected area or the affected area information to be subsequently acquired and the subject ID.

In Step 1505, the system control circuit 220 displays a user interface prompting the user to input the information about the region where the affected area exists in the affected area information in the display unit 223. Specifically, as in FIG. 8A and FIG. 8B in the first embodiment, the region selection items 801 for specifying the regions: Head, Shoulder, Arm, Back, Waist, Hip, and Leg of the affected area are displayed to cause the user to select any of them.

In Step 1506, the user inputs the information about the affected area. Then, the operation goes to Step 402. Going to the step to shoot the affected area after the information about the region of the affected area to be shot is selected in the above manner prevents wrong selection of the information about the region of the affected area.

Since the collation of the subject ID is performed in Step 1513, it is not necessary for the image processing apparatus 300 to perform the collation of the subject ID after acquiring the image data including the affected area. In addition, since the information about the region of the affected area is input in Step 1506, it is not necessary for the user to input the information about the region of the affected area in Step 1507 and Step 1508 after the image data including the affected area is acquired and it is sufficient for the user to input the evaluation value of each evaluation item in Step 1507 and Step 1508.

As described above, in the image processing system 11 according to the present embodiment, it is possible to identify and store the image data about the affected area 102 and the result of analysis of the image data for each subject and to confirm whether each evaluation item is relieved or made worse only using the imaging apparatus the user has on hand. Accordingly, the user is capable of confirming management information about the affected area that has been registered immediately after shooting the affected area only using the imaging apparatus the user has on hand. In addition, displaying the severity level that is currently confirmed in comparison with the last management information enables the user to confirm whether the symptom is relieved or made worse at first sight.

The user is capable of confirming the result of analysis of the image data about the affected area 102 in association with the subject ID and the name of the subject from the terminal apparatus 1000, such as a tablet terminal, using a Web browser or a dedicated application.

In all the embodiments described above, a process to achieve the same effects as in the work flows in FIG. 4, FIG. 9, and FIG. 11 is capable of being performed only with the imaging apparatus 200 by installing a circuit corresponding to the auxiliary arithmetic unit 317 in the imaging apparatus 200. In this case, the same effects as in the image processing systems composed of the imaging apparatus 200 and the image processing apparatus 300 described above are achieved only with the imaging apparatus 200. Receiving the new leaned model that is created in an external computer enables improvement of the accuracy of the inference process of the affected area and extraction of the affected area of a new type.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An image processing system comprising an imaging apparatus and an image processing apparatus, wherein:
the imaging apparatus includes
an imaging device configured to receive light from a subject to generate image data,
a first communication circuit configured to output the image data to a communication network, and
a display configured to display an image based on the image data generated by the imaging device,
the image processing apparatus includes
a second communication circuit configured to acquire the image data over the communication network, and
an arithmetic circuit configured to extract an affected area of the subject from the image data,
the second communication circuit outputs information indicating a result of extraction of the affected area extracted by the arithmetic circuit to the communication network,
the first communication circuit acquires the information indicating the result of extraction of the affected area over the communication network, and
the display performs display based on the information indicating the result of extraction of the affected area and display causing a user to input evaluation values of a plurality of predetermined evaluation items in the affected area.

2. The image processing system according to claim 1, wherein
the display displays an image based on the image data on which the result of extraction of the affected area is superimposed and which is used to extract the affected area by the arithmetic circuit.

3. The image processing system according to claim 1, wherein
the display displays a live view image on which the result of extraction of the affected area is superimposed and which is generated by the imaging device.

4. The image processing system according to claim 1, wherein
the arithmetic circuit generates information indicating a size of the affected area extracted from the image data, and
the second communication circuit outputs the information indicating the size generated by the arithmetic circuit to the communication network.

5. The image processing system according to claim 4, wherein
the imaging apparatus includes a generating circuit configured to generate distance information about a distance from the imaging apparatus to the subject,
the first communication circuit outputs the distance information to the communication network,
the second communication circuit acquires the distance information over the communication network, and
the arithmetic circuit generates the information indicating the size of the affected area based on the distance information.

6. The image processing system according to claim 4, wherein
the display performs display based on the information indicating the result of extraction of the affected area and the information indicating the size.

7. The image processing system according to claim 4, wherein
the information indicating the size of the affected area is at least one of lengths in at least two directions of the affected area, an area of the affected area, an area of a rectangular area circumscribed around the affected area, and a scale bar for measuring the size of the affected area.

8. The image processing system according to claim 7, wherein
the arithmetic circuit converts a size of the affected area on the image data based on information indicating an angle of view of the image data or a size of a pixel and the distance information to generate the information indicating the size of the affected area.

9. The image processing system according to claim 1, wherein
the arithmetic circuit identifies information indicating a size of the affected area for each subject having the affected area and stores the identified information in storage circuit.

10. The image processing system according to claim 9, wherein
the arithmetic circuit identifies the information indicating the size of the affected area based on the subject having the affected area and a date and time when the image data used in the extraction of the affected area is generated and stores the identified information in the storage circuit.

11. The image processing system according to claim 9, wherein
the arithmetic circuit transmits, in response to a request from an external terminal apparatus, the information indicating the size of the affected area, which corresponds to a subject specified in the request, to the terminal apparatus.

12. The image processing system according to claim 9, wherein
the second communication circuit further acquires image data including a code for identifying the subject, which is output from the first communication circuit, over the communication network, and
the arithmetic circuit extracts information identifying the subject having the affected area from the image data including the code for identifying the subject.

13. The image processing system according to claim 1, wherein
the arithmetic circuit causes second display different from the display to display the information indicating the result of extraction of the affected area.

14. The image processing system according to claim 13, wherein
the arithmetic circuit causes the second display to arrange for display an image based on the image data on which the result of extraction of the affected area is superimposed and an image based on the image data acquired by the second communication circuit.

15. The image processing system according claim 1, wherein
the display causes the user to input the evaluation values of the plurality of evaluation items in response to the acquisition of the result of extraction of the affected area.

16. An imaging apparatus comprising:
an imaging device configured to receive light from a subject to generate image data;
a communication circuit configured to output the image data to an external apparatus over a communication network; and
a display configured to display an image based on the image data generated by the imaging device, wherein
the communication circuit acquires information indicating a result of extraction of an affected area of the subject in the image data from the external apparatus over the communication network, and
the display performs display based on the information indicating the result of extraction of the affected area and display causing a user to input evaluation values of a plurality of predetermined evaluation items in the affected area.

17. The imaging apparatus according to claim 16, wherein
the display displays an image based on the image data on which the result of extraction of the affected area is superimposed and which is output to the external apparatus.

18. The imaging apparatus according to claim 16, wherein
the display displays a live view image on which the result of extraction of the affected area is superimposed and which is generated by the imaging device.

19. The imaging apparatus according to claim 16, wherein
the communication circuit acquires information indicating a size of the affected area in the image data from the external apparatus over the communication network, and
the display performs display based on the information indicating the result of extraction of the affected area and the information indicating the size.

20. The imaging apparatus according to 19, further comprising:
a generating circuit configured to generate distance information about a distance from the imaging apparatus to the subject, the imaging apparatus is wherein
the communication circuit outputs the distance information to the external apparatus over the communication network.

21. The imaging apparatus according to claim 19, wherein
the information indicating the size of the affected area is at least one of lengths in at least two directions of the affected area, an area of the affected area, an area of a rectangular area circumscribed around the affected area, and a scale bar for measuring the size of the affected area.

22. The imaging apparatus according to claim 16, wherein
the communication circuit outputs information for identifying the subject having the affected area to the external apparatus over the communication network.

23. The imaging apparatus according to claim 16, wherein
the display causes the user to input the evaluation values of the plurality of evaluation items in response to the acquisition of the result of extraction of the affected area.

24. A method of controlling an image processing system including an imaging apparatus that includes an imaging device, a display, and first communication device and an image processing apparatus that includes an arithmetic device and a second communication device, the method comprising:
receiving light from a subject to generate image data by the imaging device;
outputting the image data to a communication network by the first communication device;
acquiring the image data over the communication network by the second communication device;
extracting an affected area of the subject from the image data by the arithmetic device;
outputting information indicating a result of extraction of the affected area to the communication network by the second communication device;
acquiring the information indicating the result of extraction of the affected area over the communication network by the first communication device; and
performing display based on the information indicating the result of extraction of the affected area and display causing a user to input evaluation values of a plurality of predetermined evaluation items in the affected area by the display.

25. A method of controlling an imaging apparatus, the method comprising:
receiving light from a subject to generate image data;
outputting the image data to an external apparatus over a communication network;
acquiring information indicating a result of extraction of an affected area of the subject in the image data from the external apparatus over the communication network; and
causing a display to perform display based on the information indicating the result of extraction of the affected area and display causing a user to input evaluation values of a plurality of predetermined evaluation items in the affected area.

26. A computer-readable non-volatile storage medium storing an instruction causing a computer to perform steps of a method of controlling an imaging apparatus, the method of controlling the imaging apparatus comprising:
receiving light from a subject to generate image data;
outputting the image data to an external apparatus over a communication network;
acquiring information indicating a result of extraction of an affected area of the subject in the image data from the external apparatus over the communication network; and
causing a display to perform display based on the information indicating the result of extraction of the affected area and display causing a user to input evaluation values of a plurality of predetermined evaluation items in the affected area.

27. An imaging apparatus comprising:
an imaging device configured to receive light from a subject to generate image data;
a control circuit configured to acquire a result of extraction of a certain area of the subject in the image data; and
an interface circuit configured to cause a user to input evaluation values of a plurality of predetermined evaluation items in the certain area of the subject, wherein
the control circuit associates the evaluation values of the input plurality of evaluation items with the image data.

28. The imaging apparatus according to claim 27, wherein
the interface circuit causes the user to input the evaluation values of the plurality of evaluation items in response to the acquisition of the result of extraction of the certain area of the subject.

29. The imaging apparatus according to claim 27, wherein
the result of extraction of the certain area of the subject includes information indicating a size of the certain area.

30. The imaging apparatus according to claim 27, wherein
the interface circuit causes the user to input information about a region of the subject in which the certain area exists.

31. The imaging apparatus according to claim 30, wherein
the interface circuit causes the user to input the information about the region in which the certain area exists before the result of extraction of the certain area is acquired.

32. The imaging apparatus according to claim 27, wherein
the interface circuit displays the evaluation items for which the evaluation values are input and the evaluation items for which the evaluation values are not input, among the plurality of evaluation items, in different modes.

33. The imaging apparatus according to claim 27, wherein
the control circuit acquires information for identifying the subject having the certain area and associates the image data from which the certain area is extracted with the evaluation values of the plurality of evaluation items for each subject.

34. The imaging apparatus according to claim 33, wherein
the control circuit acquires the evaluation values of the plurality of evaluation items that has been associated with the same subject.

35. The imaging apparatus according to claim 34, wherein
the interface circuit displays the evaluation values of the plurality of evaluation items that are newly acquired and the evaluation values of the plurality of evaluation items that have been acquired.

36. The imaging apparatus according to claim 33, wherein
the interface circuit displays a result of identification of the subject having the certain area.

37. The imaging apparatus according to claim 27, further comprising:
a communication circuit configured to transmit the image data generated by the imaging device to an image processing apparatus, which is an external apparatus, over a communication network and receiving information about a result of extraction of the certain area from the image data from the image processing apparatus over the communication network.

38. The imaging apparatus according to claim 37, wherein
the communication circuit transmits the image data and distance information from the imaging apparatus to the subject to the image processing apparatus over the communication network and receives the information about the result of extraction of the certain area, which includes information indicating a size of the certain area, from the image processing apparatus over the communication network.

39. The imaging apparatus according to any of claim 27, wherein
the certain area is an affected area.

40. A method of controlling an imaging apparatus, the method comprising:
receiving light from a subject to generate image data;
acquiring a result of extraction of a certain area of the subject in the image data;
causing a user to input evaluation values of a plurality of predetermined evaluation items in the certain area of the subject; and
associating the evaluation values of the input plurality of evaluation items with the image data.

41. A computer-readable non-volatile storage medium storing an instruction causing a computer to perform steps of a method of controlling an imaging apparatus, the method of controlling the imaging apparatus comprising:
receiving light from a subject to generate image data;
acquiring a result of extraction of a certain area of the subject in the image data;
causing a user to input evaluation values of a plurality of predetermined evaluation items in the certain area of the subject; and
associating the evaluation values of the input plurality of evaluation items with the image data.

42. An electronic device comprising:
a communication circuit configured to acquire image data generated by an imaging apparatus and information indicating evaluation values of a plurality of evaluation items for an affected area of a subject in the image data, which is input by a user with the imaging apparatus, over a communication network; and
a control circuit configured to cause a display to display an image based on the image data and the evaluation values of the plurality of evaluation items.

43. The electronic device according to claim 42, wherein
the control circuit causes the display to identify for display an image based on the image data and the evaluation values of the plurality of evaluation items based on the subject having certain area and a date and time when the image data used in extraction of the certain area is generated.

44. A method of controlling an electronic device, the method comprising:
acquiring image data generated by an imaging apparatus and information indicating evaluation values of a plurality of evaluation items for an affected area of a subject in the image data, which is input by a user with the imaging apparatus, over a communication network; and
causing a display to display an image based on the image data and the evaluation values of the plurality of evaluation items.

45. A computer-readable non-volatile storage medium storing an instruction causing a computer to perform steps of a method of controlling an electronic device, the method of controlling the electronic device is characterized by comprising:
acquiring image data generated by an imaging apparatus and information indicating evaluation values of a plurality of evaluation items for an affected area of a subject in the image data, which is input by a user with the imaging apparatus, over a communication network; and
causing a display to display an image based on the image data and the evaluation values of the plurality of evaluation items.
